(19) United States
(12) Patent Application Publication
(10) Pub. No.: US 2009/0182019 A1
(43) Pub. Date: Jul. 16, 2009

(54) Histone deacetylase inhibitors

(75) Inventors: Robert A. Casero, Glen Arm, MD (US); Patrick M. Woster, Canton, MI (US); Sheeba Varghese, Detroit, MI (US)

Correspondence Address: EDWARDS ANGELL PALMER & DODGE LLP, P.O. BOX 55874, BOSTON, MA, 02205, US

(73) Assignees: The Johns Hopkins University, Baltimore, MD (US); Wayne State University, Detroit, MI (US)

(21) Appl. No.: 11/918,849

(22) PCT Filed: Apr. 18, 2006
(86) PCT No.: PCT/US06/14375

§ 371 Date: Feb. 2, 2009

Related U.S. Application Data
(60) Provisional application No. 60/672,470, filed on Apr. 18, 2005.

(51) Int. Cl. A61K 31/4406; C07C 237/20; A61K 31/165; C07D 213/56; A61P 35/00; C12N 9/99

(52) U.S. Cl. 514/357; 564/163; 514/619; 514/620; 564/164; 546/335; 435/184

(57) ABSTRACT

The present invention provides novel HDAC inhibitors and methods of treating diseases using the same.

### CROSS-REFERENCES TO RELATED APPLICATIONS

This application claims the benefit of U.S. Provisional Patent Application No. 60/672,470, filed Apr. 18, 2005, the content of which are herein incorporated by reference in their entirety for all purposes.

### STATEMENT AS TO RIGHTS TO INVENTIONS MADE UNDER FEDERALLY SPONSORED RESEARCH OR DEVELOPMENT

This invention was made with government support under CA85509 awarded by the National Institutes of Health. The government has certain rights to the invention.

### BACKGROUND OF THE INVENTION

The reversible acetylation of histones, mediated by histone acetyltransferases (HAT) and histone deacetylases (HDAC) plays a critical role in transcriptional regulation. The balance between the acetylated and deacetylated forms of histones associated with DNA control the degree of expression of various cellular proteins by modulating the level of transcription. In some tumor cell types, excessive deacetylation of histones is thought to be associated with aberrant transcription of p53, and results in underexpression of tumor suppressor factors such as p21, leading to rapid growth of transformed cells and tumor progression. Histone hyperacetylation caused by HDAC inhibitors such as trichostatin (TSA, 1) and MS-275 (2) (FIG. 1) can cause growth arrest of a wide range of transformed cells, and can inhibit the growth of human tumor xenografts. Although they are effective both in vitro and in vivo, HDAC inhibitors typified by TSA and MS-275 suffer from lack of specificity among the various forms of HDAC, and unacceptable toxicity to non-cancerous cells. Thus it would be desirable to identify potent HDAC inhibitors that restore the expression of normal tumor suppressor factors without producing significant dose-limiting toxicity.

The present invention addresses these and other needs in the art.

### BRIEF SUMMARY OF THE INVENTION

It has been discovered that polyamino-Zinc metal binding compounds are potent inhibitors of HDAC proteins.

In one aspect, the present invention provides histone deacetyalse (HDAC) inhibitors including Zn⁺² binding moiety covalently bonded to a polyamino moiety (e.g. a spermine- or spermidine-like moiety).

In another aspect, the present invention provides a method of decreasing the catalytic activity of histone deacetylase. The method includes contacting the histone deacetylase with an HDAC inhibitor of the present invention.

In another aspect, the present invention provides a method of increasing cellular reexpression of p21. The method includes contacting a cell with an HDAC inhibitor of the present invention.

In another aspect, the present invention provides a method of decreasing cellular proliferation. The method includes contacting a cell, or plurality of cells, with an HDAC inhibitor of the present invention.

In another aspect, the present invention provides a method of treating cancer in a subject in need thereof. The method includes administering to the subject a therapeutically effective amount of an HDAC inhibitor.

In another aspect, the present invention provides a pharmaceutical composition including an HDAC inhibitor and a pharmaceutically acceptable excipient.

### BRIEF DESCRIPTION OF THE DRAWINGS

FIG. 1. Structures of trichostatin, MS-275, spermidine and spermine.

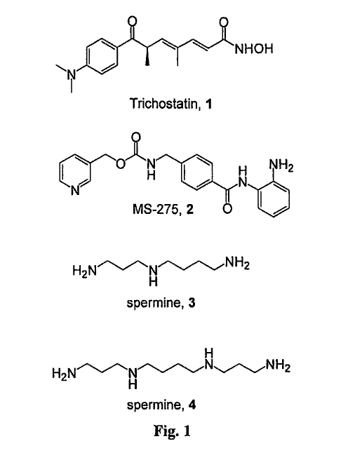

FIG. 2. In vitro percent inhibition of HDAC caused by trichostatin and a series of novel polyaminohydroxamates. The enzyme preparation was exposed to a 1 μM concentration of each inhibitor as described in the Experimental section. Each data point is the average of three determinations that differed by 3% or less in each case.

FIG. 3. In vitro dose-response for inhibition of HDAC caused by trichostatin and polyaminohydroxamates 11, 14 and 19. The enzyme preparation was exposed to a concentration range of each inhibitor as described in the Experimental section. Each data point is the average of three determinations that differed by 3% or less in each case.

FIG. 4. Toxicity of MS-275, TSA and compound 19 to ML-1 myelocytic leukemia cells in culture after 3 days of exposure. Cells were exposed to a range of concentrations of the inhibitor, and cell viability was determined using an MTT assay. Each data point is the result of 3 separate determinations which differed by less than 3% in all cases.

FIG. 5. Toxicity of MS-275, TSA and compound 19 to ML-1 myelocytic leukemia cells in culture after 7 days of exposure. Cells were exposed to a range of concentrations of the inhibitor, and cell viability was determined using an MTT assay. Each data point is the result of 3 separate determinations which differed by less than 3% in all cases.

FIG. 6. Toxicity of MS-275, TSA and compound 19 to ML-1 myelocytic leukemia cells in culture after 3 days of exposure. Cells were exposed to a range of concentrations of the inhibitor, and cell viability was determined by direct cell count. Each data point is the result of 3 separate determinations which differed by less than 3% in all cases.

FIG. 7. Toxicity of MS-275, TSA and compound 19 to ML-1 myelocytic leukemia cells in culture after 7 days of exposure. Cells were exposed to a range of concentrations of the inhibitor, and cell viability was determined by direct cell count. Each data point is the result of 3 separate determinations which differed by less than 3% in all cases.

FIG. 8. Acetylation of histone H3 and H4 and expression of p21^{WAF1/CIP1} in ML-1 cells. ML-1 cells were incubated with indicated compounds prior to Western blot analysis. MS-275 was used as a positive control.

FIG. 9. Effect of linker chain length on inhibition of HDAC.

FIG. 10. Inhibitory activity of selected certain HDAC inhibitors against HDAC 1 (left), 4 (center) or 6 (right). Each data point is the average of three determinations which in every case differed by less than 3%.

### DETAILED DESCRIPTION OF THE INVENTION

### I. Definitions and Abbreviations

Abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—, —CH₂CH═CHCH₂—, —CH₂C═CCH₂—, —CH₂CH₂CH(CH₂CH₂CH₃)CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent radicals of aryl and heteroaryl, respectively.

For brevity, the term “aryl” when used in combination with other terms (e.g., aryloxo, arylthioxo, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those allyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” referrers to a carbon or heteroatom.

The term “oxo” as used herein means an oxygen that is double bonded to a carbon atom.

Each of above terms (e.g., “alkyl,” “heteroalkyl,” “cycloalkyl, and “heterocycloalkyl”, “aryl,” “heteroaryl” as well as their divalent radical derivatives) are meant to include both substituted and unsubstituted forms of the indicated radical. Preferred substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

An “aminoalkyl” as used herein refers to an amino group covalently bound to an alkylene linker. The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The compounds of the present invention may exist as salts. The present invention includes such salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” in reference to a particular disease includes prevention of the disease.

The symbol [symbol] denotes the point of attachment of a moiety to the remainder of the molecule.

### II. Histone Deacetylase Inhibitors

In one aspect, the present invention provides histone deacetyalse (HDAC) inhibitors (also referred to herein as “polyamino histone deacetyalase inhibitors (PAHIs),” or “the compounds of the present invention”). The HDAC inhibitors include a polyamino portion and a Zn⁺² binding moiety. Without being bound by any particular mechanistic functional theory, in some embodiments, the compounds enter cells using the polyamine cellular transport system, and may be directed to histones and DNA by virtue of the polyamine portion of the structure. In some embodiments, the compound is a polyamino hydroxamic acid (PAHA), wherein the Zn⁺² binding moiety is a hydroxamic acid. In other embodiments, the compound is a polyaminobenzamides (PABA), wherein the Zn⁺² binding moiety is a benzamide. A “Zn⁺² binding moiety” as used herein refers to a chemical group capable of forming a chemical bond (e.g. metal coordination, metal chelation, etc.) with a Zn⁺² ion.

In a preferred embodiment, the compounds of the present invention have the formula:

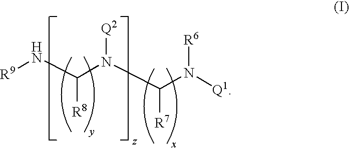

In Formula (I), Q¹ and Q² are independently hydrogen or a moiety having the formula

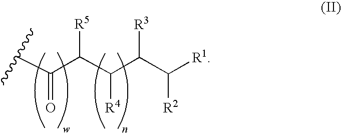

In Formulae (I) and (II), n represents an integer from 1 to 20, and w is an integer from 0 to 1. The symbols x and y are independently integers from 1 to 20, and z is an integer from 0 to 10.

R¹ is a Zn⁺²-binding moiety. R², R³, R⁴, and R⁵ are independently hydrogen or unsubstituted C₁-C₆ alkyl. R⁶ is hydrogen or -L¹-R¹⁰. L¹ is a bond, substituted or unsubstituted alkylene, or substituted or unsubstituted heteroalkylene. R¹⁰ is hydrogen, substituted or unsubstituted heterocycloalkyl, substituted or unsubstituted cycloalkyl, substituted or unsubstituted heteroaryl, or substituted or unsubstituted aryl. R⁷ and R⁸ are independently hydrogen, —NH₂, or unsubstituted C₁-C₆ alkyl. R⁹ is hydrogen or -L²-R¹¹. L² is a bond, substituted or unsubstituted alkylene, or substituted or unsubstituted heteroalkylene. R¹¹ is independently hydrogen, substituted or unsubstituted heterocycloalkyl, substituted or unsubstituted cycloalkyl, substituted or unsubstituted heteroaryl, or substituted or unsubstituted aryl.

The compounds of the present invention include at least one Zn⁺²-binding moiety (R¹). Thus, in some embodiments, if z is 0 then Q¹ is not hydrogen. In some embodiments, if Q¹ is hydrogen then at least one Q² is not hydrogen and z is an integer from 1 to 10. In some embodiments, only one of Q¹ or Q² is not hydrogen.

In other embodiments, if R⁶ and R¹⁰ are hydrogen, then at least one of R⁹ or R¹¹ are not hydrogen, and if R⁹ and R¹¹ are hydrogen, then at least one of R⁶ or R¹⁰ are not hydrogen.

A variety of Zn⁺²-binding moieties are useful as R¹ moieties. In some embodiments, R¹ is a hydroxamic acid or a substituted or unsubstituted benzamide. In other embodiments, R¹ is —C(O)NHOH, —C(O)OH, —C(O)NH-(2-amino-phenyl), or substituted or unsubstituted tetrazolyl.

The symbol n may be an integer from 1 to 10, from 1 to 5, or from 2 to 5. In some embodiments, w is 1.

In some embodiments, z is at least 1. The symbol z may also be an integer from 1 to 5, or from 1 to 2. In some embodiments, x and y are integers from 1 to 10, from 1 to 5, or from 2 to 5. The symbols x and y may also represent integers from 3 to 4. In other embodiments, x is 3 and y is 4.

The symbol y may independently be from 1 to 10, from 1 to 5, from 2 to 5, 3 or 4. Thus, one of skill will understand that where z is greater than 1, each iteration of y is optionally different.

In some embodiments, the HDAC inhibitor includes at least two protonated nitrogens that form part of the polyamino portion of the inhibitor. In some embodiments, the number of protonated nitrogens is two or three. Thus, in certain embodiments, at least one of R⁶, R⁷, Q², or R⁸ is hydrogen. In other embodiments, one or two of R⁶, R⁷, Q², of R⁸ is hydrogen. And in certain other embodiments, R², R³, R⁴, R⁵, R⁷ and R⁸ are hydrogen.

In some embodiments, R⁶ is hydrogen. R⁹ may be -L²-R¹¹, where L² is substituted or unsubstituted alkylene, and R¹¹ is substituted or unsubstituted aryl. L² may also be substituted or unsubstituted C₁-C₅ alkylene. In other embodiments, L² is unsubstituted C₁-C₅ alkylene or C₁-C₅ alkylene substituted with substituted or unsubstituted aryl. L² may also be unsubstituted C₁-C₅ alkylene or C₁-C₅ alkylene substituted with unsubstituted aryl. In certain embodiments, L² is —CH₂—CH₂—CH(phenyl)-.

R⁶ may also be -L¹-R¹⁰. L¹ may be substituted or unsubstituted alkylene (e.g. methylene, ethylene, propylene etc.), or substituted or unsubstituted heteroalkylene. In some embodiments where L¹ is a substituted or unsubstituted heteroalkylene, the heteroalkylene contains at least one protonated hydrogen. Thus, the heteroalkylene may be an alkylene containing a protonated nitrogen heteroatom (e.g. —(C₁-C₁₀)alkylene-NH— or —(C₁-C₁₀)alkylene-SH—).

In some embodiments, R¹⁰ is substituted or unsubstituted heterocycloalkyl, substituted or unsubstituted cycloalkyl, substituted or unsubstituted heteroaryl, or substituted or unsubstituted aryl. R¹⁰ may also be substituted or unsubstituted cycloalkyl, or substituted or unsubstituted aryl (e.g. phenyl). R¹⁰ may be unsubstituted aryl or aryl substituted with one or more of the following: unsubstituted C₁-C₁₀ alkyl, unsubstituted aryl, or —NR¹²R¹³. R¹² and R¹³ are independently hydrogen or unsubstituted C₁-C₁₀ alkyl.

R⁷ and R⁸ may be hydrogen or unsubstituted C₁-C₆ alkyl.

R⁹ may be -L²-R¹¹. L² may be substituted or unsubstituted alkylene (e.g. methylene, ethylene, propylene etc.), or substituted or unsubstituted heteroalkylene. Where L is a substituted or unsubstituted heteroalkylene, the heteroalkylene may be an alkylene with a sulfhydryl or protonated nitrogen group (e.g. —(C₁-C₁₀)alkylene-NH— or —(C₁-C₁₀)alkylene-SH—). R¹¹ may be unsubstituted cycloalkyl (e.g. cyclopropyl cyclobutyl, cyclopentyl, cyclohexyl, or cycloheptyl) unsubstituted aryl or aryl substituted with one or more of the following: unsubstituted C₁-C₁₀ alkyl, unsubstituted aryl, or —NR¹²R¹³. R¹² and R¹³ are independently hydrogen or unsubstituted C₁-C₁₀ alkyl.

In some embodiments, each substituted group described above in the compounds of Formulae (I) and (II) is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, and/or substituted heteroalkylene, described above in the compounds of Formulae (I) and (II) are substituted with at least one substituent group. In other embodiments, at least one or all of these groups are substituted with at least one size-limited substituent group. Alternatively, at least one or all of these groups are substituted with at least one lower substituent group.

In other embodiments of the compounds of Formulae (I) and (II), each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl, each substituted or unsubstituted alkylene is a substituted or unsubstituted C₁-C₂₀ alkylene, and/or each substituted or unsubstituted heteroalkylene is a substituted or unsubstituted 2 to 20 membered heteroalkylene.

Alternatively, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, each substituted or unsubstituted heterocycloalkyl is a substituted or unsubstituted 5 to 7 membered heterocycloalkyl, each substituted or unsubstituted alkylene is a substituted or unsubstituted C₁-C₈ alkylene, and/or each substituted or unsubstituted heteroalkylene is a substituted or unsubstituted 2 to 8 membered heteroalkylene.

In another embodiment, the compounds of the present invention include one or more compounds of Table 1.

### III. Exemplary Syntheses

The compounds of the invention are synthesized by an appropriate combination of generally well known synthetic methods. Techniques useful in synthesizing the compounds of the invention are both readily apparent and accessible to those of skill in the relevant art. The discussion below is offered to illustrate how, in principle, to gain access to the compounds claimed under this invention and to give details on certain of the diverse methods available for use in assembling the compounds of the invention. However, the discussion is not intended to define or limit the scope of reactions or reaction sequences that are useful in preparing the compounds of the present invention.

Depending on structure, one of 5 synthetic routes was used to produce compounds 5-20, as outlined in Schemes 1-5. The structures and molecular weights of analogues 5-20 are summarized in Table 1. The synthesis of compounds 5, 7, 10, 12, 16 and 17 is shown in Scheme 1. Commercially available N-(3-bromobutyl)phthalimide 21 was first converted to the corresponding azide 22 (NaN₃, DMF), which was immediately reduced to the corresponding amine 23 by catalytic hydrogenation (10% Pd/C, 25 psi). The desired aralkyl group was then added by reductive amination of the appropriate aldehyde (NaCNBH₃, AcOH) to afford the phthalimide-protected diamines 24a-c. The secondary amine was then N-Boc protected ((Boc)₂, NaHCO₃, NaCl) to provide 25a-c, followed by removal of the phthalimide (methanolic NH₂NH₂) to give 26a-c. The free primary amine was then coupled to acid chloride 27a (n=1) or 27b (n=3), yielding 28a-e. The methyl ester in 28a-e was cleaved (IN LiOH), resulting in the free acids 29a-e, and these intermediates were then converted to hydroxamic acids 30a-e in a two step process involving formation of an activated mixed anhydride (ethylchloroformate, THF) followed by addition of hydroxylamine (1.76 M NH₂OH in MeOH). Removal of the N-Boc protecting group (20% TFAA in CH₂Cl₂) then afforded compounds 5, 7, 12, 16 and 17 as trifluoroacetate salts. During the reductive alkylation step, one of the isolated products was tertiary amine 24d, which resulted from addition of two equivalents of 2-(phenyl)benzaldehyde. Elaboration of this intermediate as described above resulted in the formation of target analogue 10.

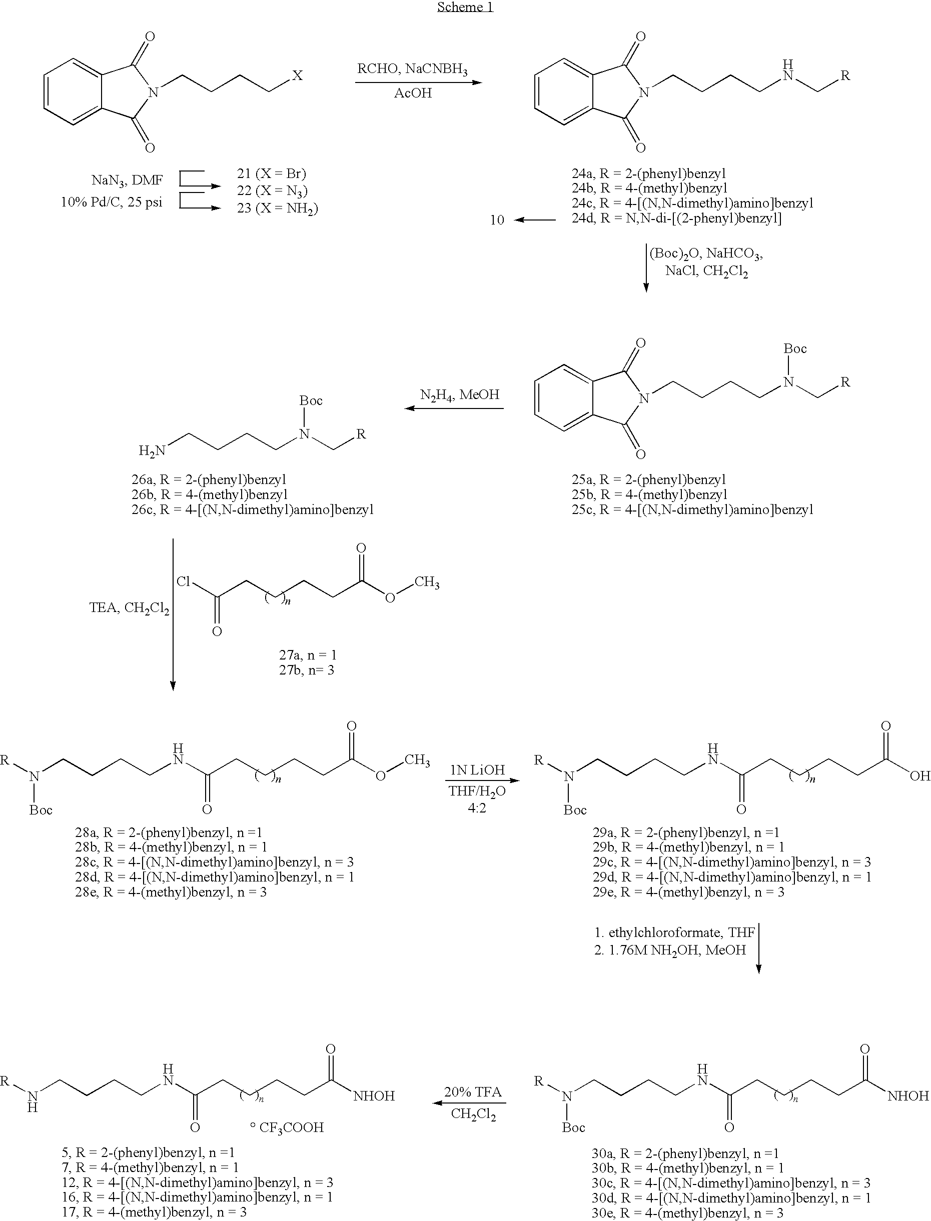

The synthetic route used to produce target analogues 11, 15, 19 and 20 is outlined in Scheme 2. The previously described dimesitylated phthalimide 31 was monoalkylated with a suitable aralkyl halide (Nall, DMF) to produce 32a-b, and subsequent removal of the phthalimide (methanolic NH₂NH₂) provided the free amines 33a-b. Coupling with 27a or 27b as described above afforded intermediates 34a-d, followed by ester cleavage (1N LiOH) to give 35a-d. These intermediates were then converted to the corresponding hydroxamic acids 36a-d as described above, and removal of the mesityl protecting groups (30% HBr in AcOH) afforded target compounds 11, 15, 19 and 20 as dihydrobromide salts. Direct deprotection of carboxylic acid 35a (30% HBr in AcOH) produced target compound 13.

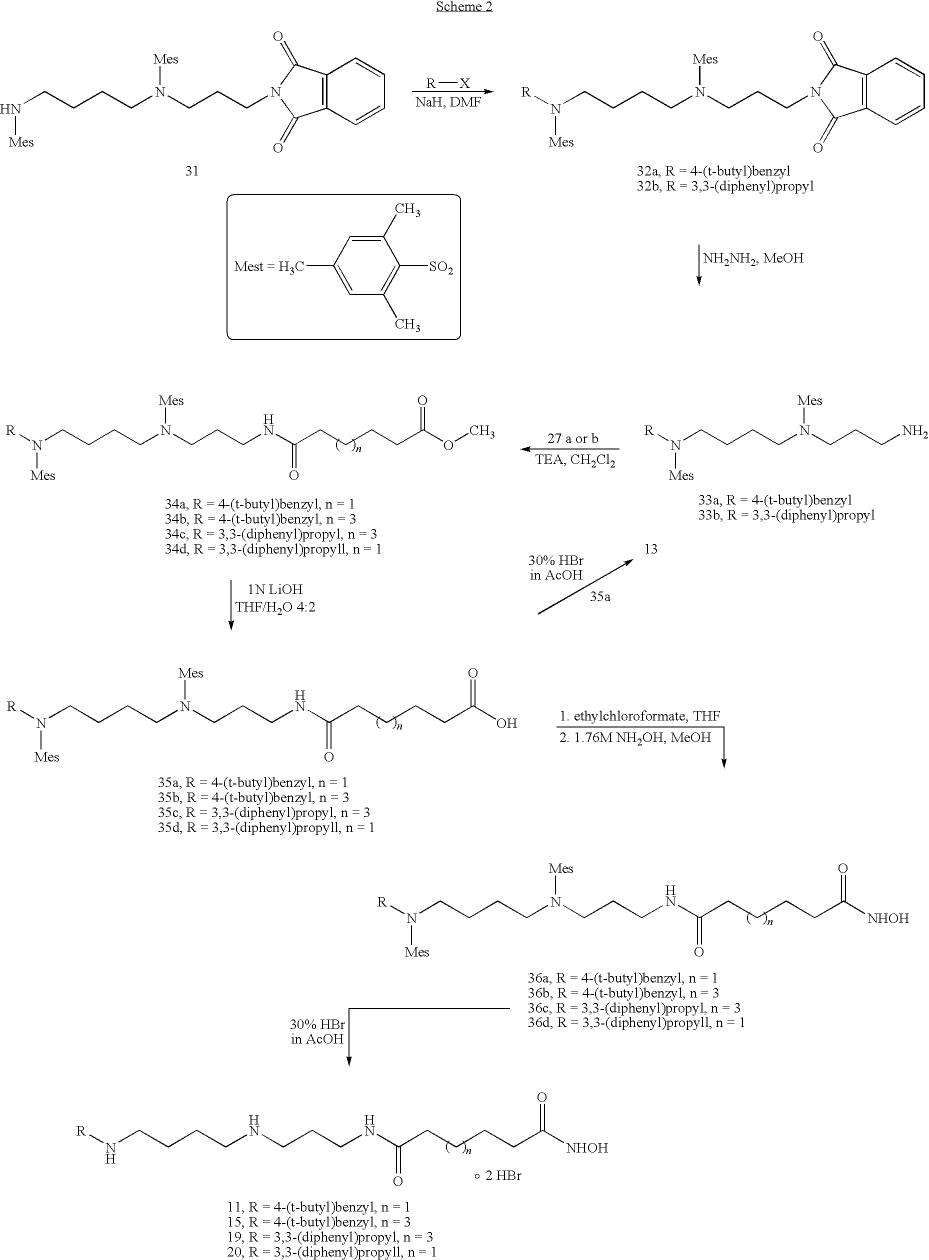

The synthesis of compounds 6, 8 and 9 is shown in Scheme 3. Tetramesitylnorspermine 37 was monoalkylated (1.1 equiv. N-(3-bromobutyl)phthalimide 21, NaH, DMF) to give 38, followed by a second alkylation with the appropriate alkyl- or aralky halide (NaH, DMF) to provide 39a-c. Removal of the phthalimide (methanolic NH₂NH₂) followed by coupling to acid chloride 27a as described above then yielded the fully protected intermediates 40a-c. Conversion of the ester in 40a-c was then accomplished in three steps as described above, resulting in hydroxamates 41a-c. Deprotection (30% HBr in AcOH) then afforded compounds 6, 8 and 9 as tetrahydrobromide salts.

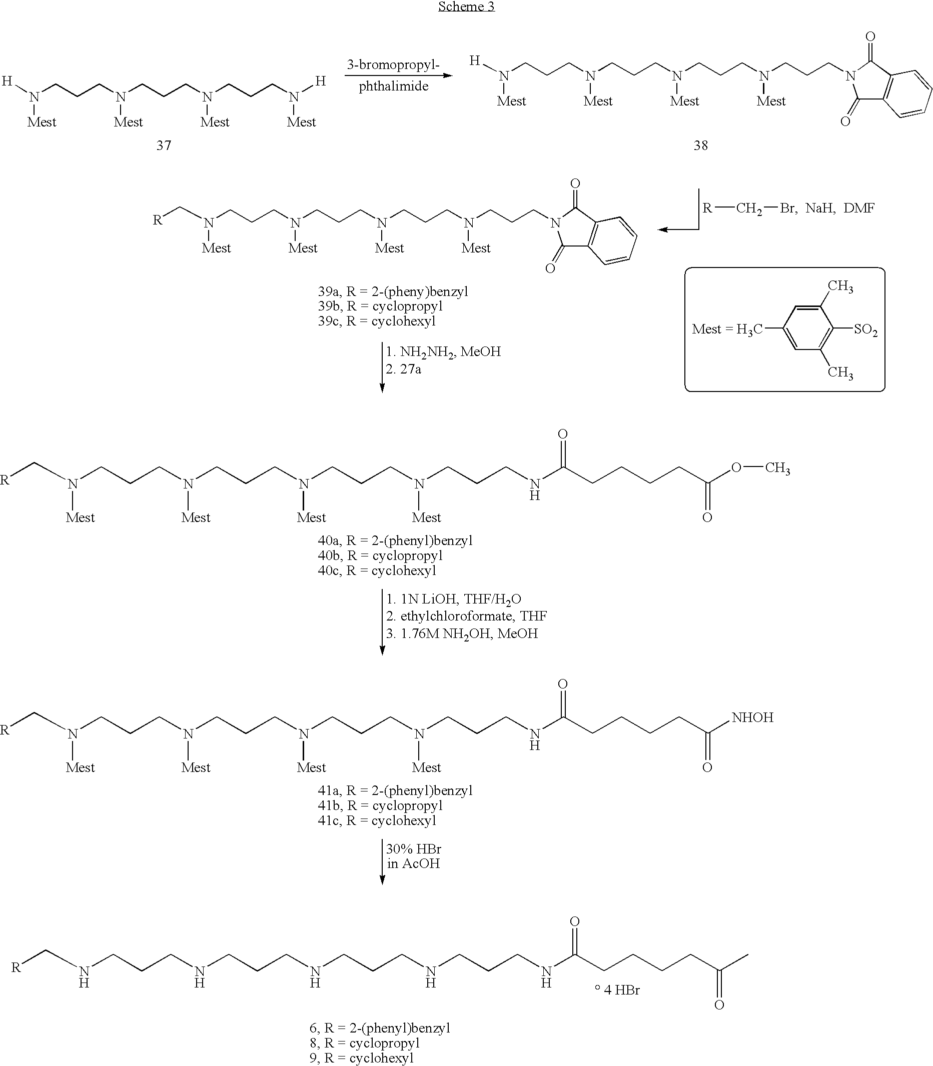

The synthesis of compound 18 is outlined in Scheme 4. Phthalimide 23 was converted to intermediate 42 by reductive animation (4-dimethylaminobenzaldehyde, NaCNBH₃, AcOH), followed by coupling to acid chloride 27b as described above to provide compound 43. Removal of the phthalimide (methanolic NH₂NH₂) followed by N-Boc protection afforded 44, and subsequent conversion of the ester to the corresponding hydroxamate in three steps as described above to give 45. Removal of the N-Boc group (20% TFAA in CH₂Cl₂) then afforded the desired 18 as the trifluoroacetate salt.

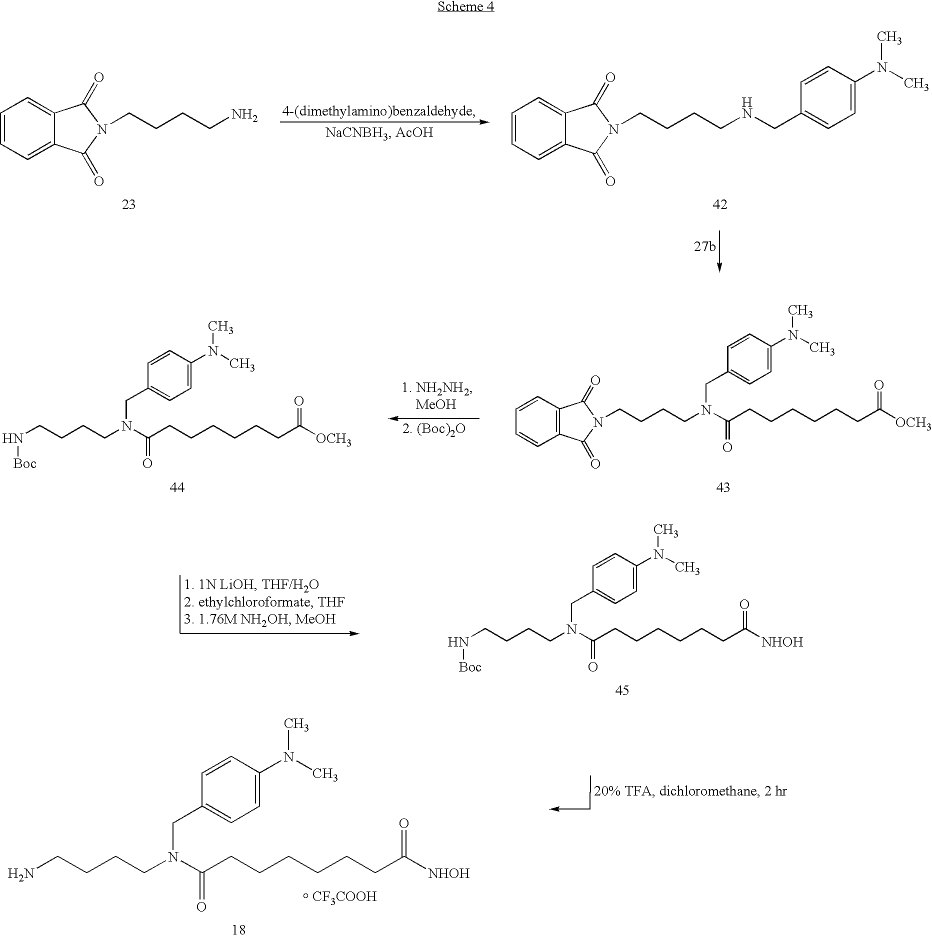

The synthesis of analogue 14 is described in Scheme 5. Removal of the phthalimide from compound 31 and reductive amination (4-dimethylaminobenzaldehyde, NaCNBH₃, AcOH) resulted in 46, which was then coupled to acid chloride 27a to afford intermediate 47. The ester was converted to the corresponding hydroxamic acid 48 as described above, and then removal of the mesityl protecting groups (30% HBr in AcOH) resulted in the formation of analogue 14 as the dihydrobromide salt.

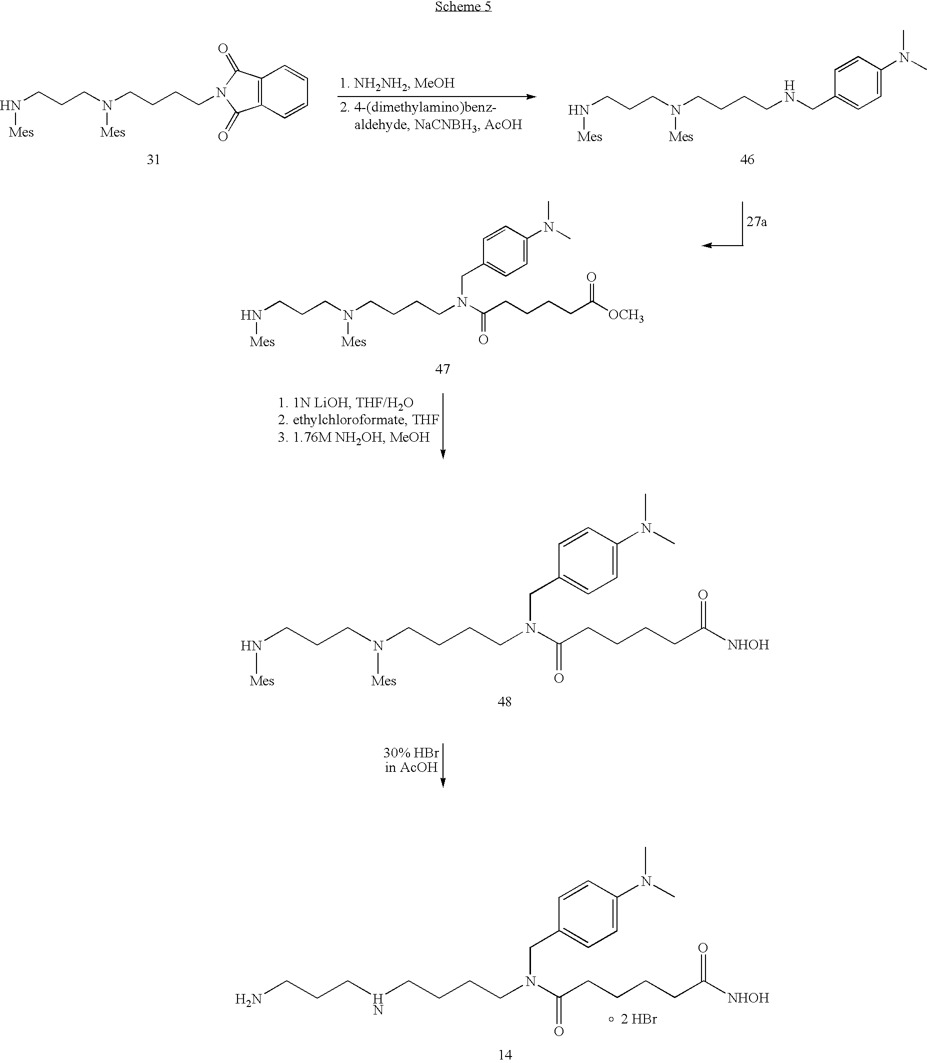

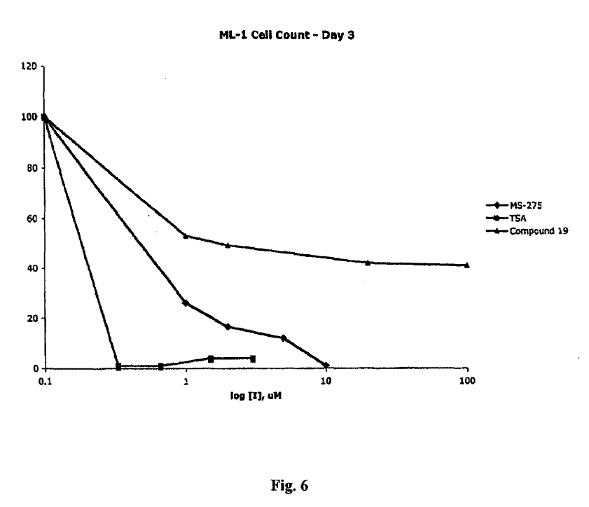

A series of polyaminobenzamide (PABA) compounds, which are also shown in FIG. 6. Some synthetic routes useful in producing PABA compounds, which contain the benzamide Zn⁺² binding moiety is outlined below.

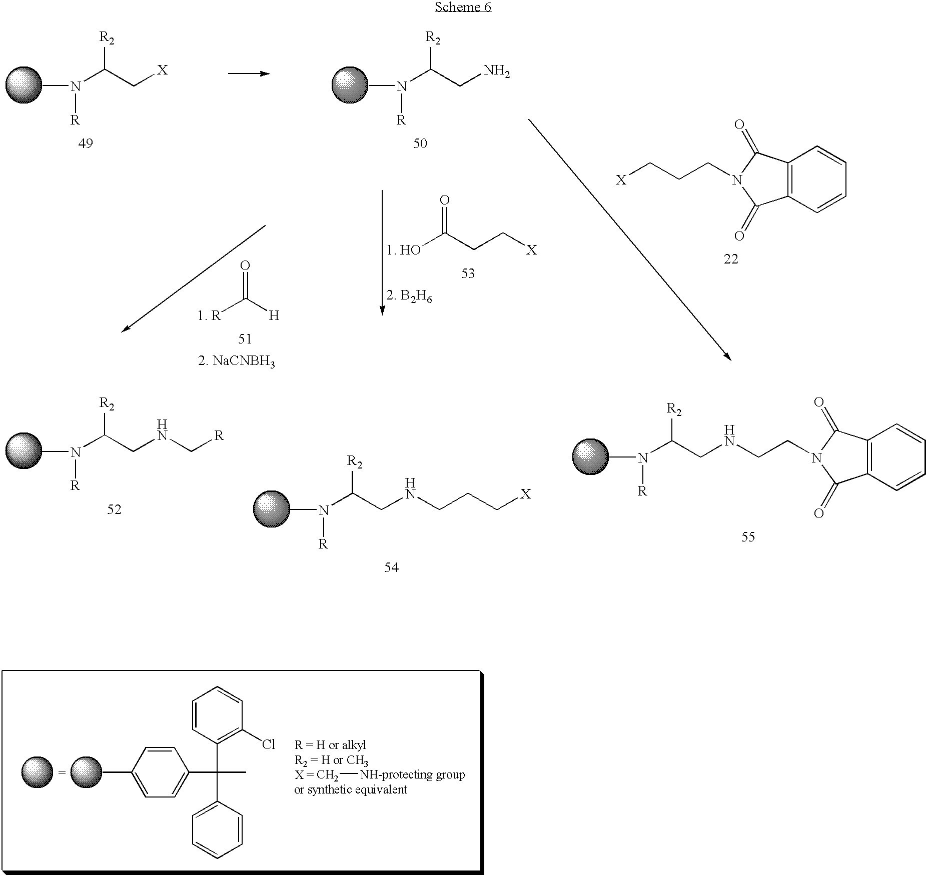

A synthetic route used to produce PABA analogues is shown in Scheme 7. p-(Aminomethyl)benzoic acid 56 is N-Boc protected (Keller et al., Org. Syn., 1985, 63, 160-171) to form 57, which is coupled to o-nitroaniline to form 58, a synthetic equivalent for the benzamide nucleus. The N-Boc protecting group is removed (HCl in CH₂Cl₂) (Keller et al., Org. Syn., 1985, 63, 160-171), and the resulting primary amine is coupled to compound 36b (Scheme 2) under peptide coupling conditions (EDCl, HOBT, triethylamine) (Woster et al., J. Med. Chem., 1989, 32, 1300-1307). Reduction of the nitro (SnCl₂, ammonium acetate) (Suzuki et al., J Med Chem 1999, 42, (15), 3001-3003) followed by removal of the mesityl protecting groups (30% HBr in AcOH) (Yajima et al., Chem. Pharm. Bull. 1978, 26, 3752-3757; Roemmele, R. C. and Rappoport, H., J. Org. Chem. 1988, 53, 2367-2371) then gives the desired PABA 59. A second PABA was made by removing the N-Boc protecting group from 58 (Keller et al., Org. Syn., 1985, 63, 160-171), followed by direct coupling of the resulting primary amine to compound 60 (EDCl, HOST, triethylamine) (Woster et al., J. Med. Chem., 1989, 32, 1300-1307) and removal of the mesityl protecting group to afford PABA 61. Compound 60 was made in turn by coupling compound 34a (Scheme 2) with 4-bromobutyric acid. As was the case with the PAHA analogues, considerable structural diversity can be introduced by variations in the polyamine chain structure, as outlined in Schemes 1-5. A series of benzamides in which the innermost phenyl group is deleted can be produced by directly coupling 36b or its homologues to o-nitroaniline.

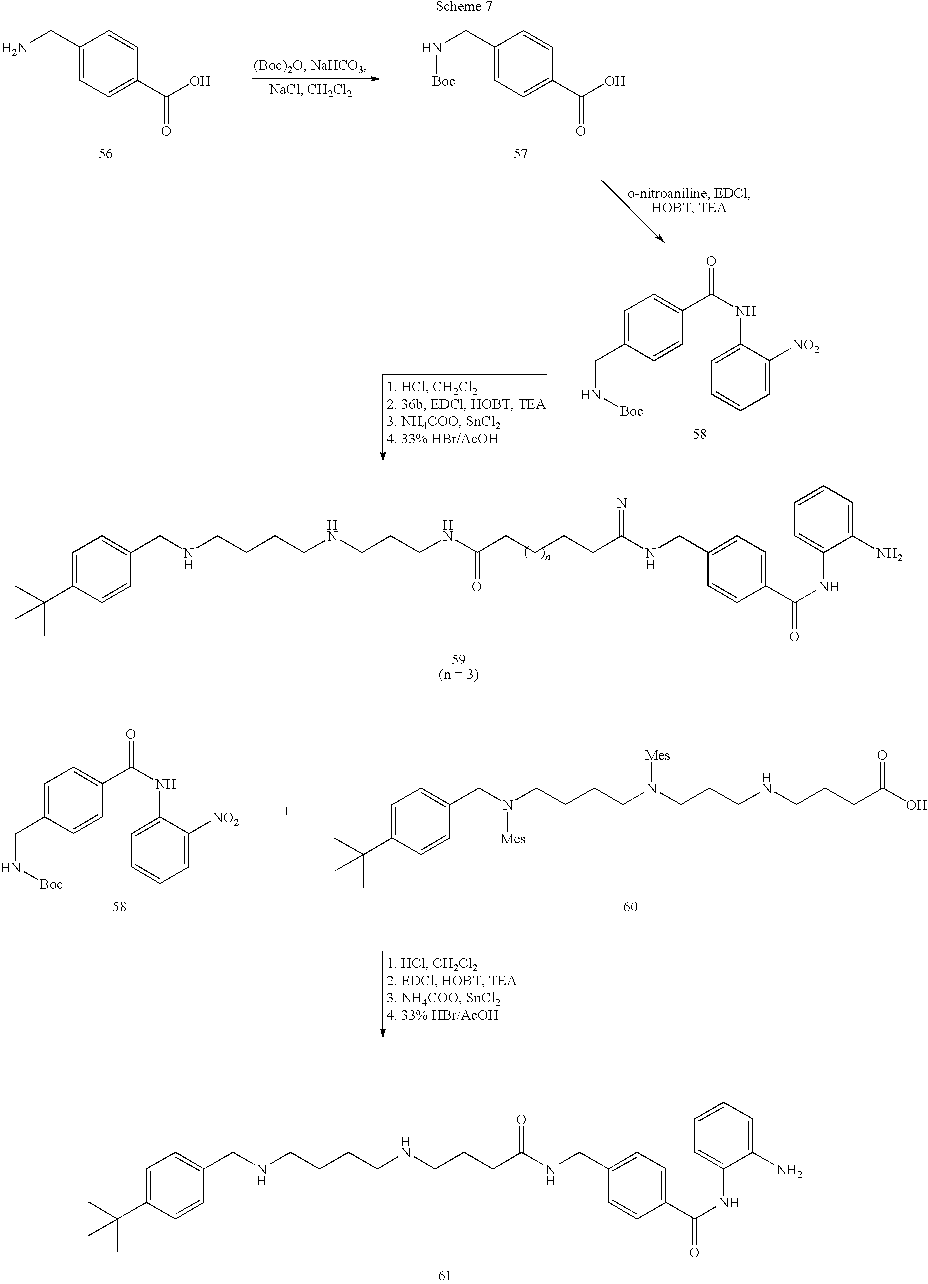

The synthesis of ³H-labelled PAHAs can be accomplished as described in Scheme 8. Thus, compound 27a (Scheme 1) can be coupled with any number of α,β-unsaturated mixed ester/acid chlorides typified by 62a and 62b using the method described in Scheme 1 (Kolhatkar et al., J. Med. Chem. 2003, 46, 2205-2215). These unsaturated esters are either available commercially, or can be readily synthesized in one step from commercial materials (Yoshikai et al., Chem. Pharm. Bull. 2005, 53, 586-588). Elaboration of the resulting protected aminoester 63a as described in Scheme 1 then results in an unsaturated homologue of the desired inhibitor, 64a. Hydrogenation of 64a (Woo et al., J. Med. Chem. 2002, 45, 2877-2885) using 10% Pd/C and tritium gas in methanol gives the ³H-form of 6. This method is readily applicable to the production of any PAHA target molecules, and offers the additional advantage of incorporating the ³H-label in the final step.

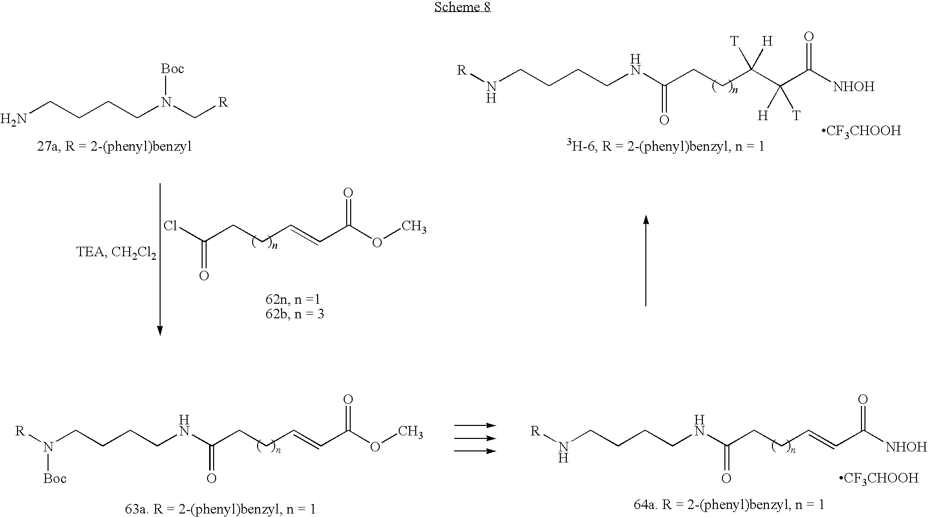

A similar synthetic strategy can be used to produce ³H-labelled PABA analogues, as outlined in Scheme 9. The α,β-unsaturated mixed ester/acid chlorides typified by 62a and 62b (Yoshikai et al., Chem. Pharm. Bull. 2005, 53, 586-588) can be coupled to a variety of amines, such as 34b (from Scheme 2), using the method described above (Kolhatkar et al., J. Med. Chem. 2003, 46, 2205-2215; Chalis and Chalis, 1970), affording analogues similar to 65b. Coupling of 65b to the synthetic equivalent 58, followed by reduction of the nitro group and removal of the mesityl protecting groups yields 66, the α,β-unsaturated homologue of target molecule 59. Catalytic hydrogenation of 66 as described above (Woo et al., J. Med. Chem. 2002, 45, 2877-2885, 10% Pd/C, ³H₂, McOH) gives the ³H-form of 59. For all ³H-labelled products, specific activity expressed in dpm/mol is determined by scintillation counting.

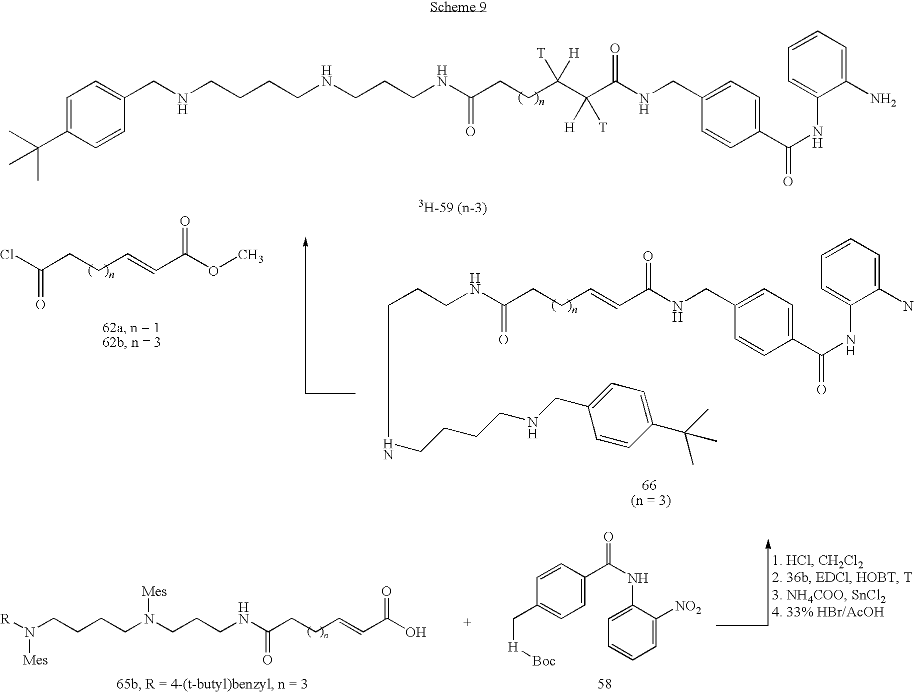

### IV. Methods

In another aspect, the present invention provides a method of decreasing the catalytic activity of histone deacetylase. The method includes contacting the histone deacetylase with an HDAC inhibitor of the present invention. The amount of HDAC inhibitor is effective to decrease catalytic activity (i.e. an effective amount). One skilled in the art will recognize that the decrease in catalytic activity is a decrease relative to the activity in the absence of the HDAC inhibitor. As used herein, “inhibit,” “inhibitor,” or “inhibition” when used in the context of histone deacetylase activity refers to a decrease in HDAC catalytic activity, and includes elimination of catalytic activity. In some embodiments, the HDAC inhibitors of the present invention selectively reduce the catalytic activity of one or more specific HDAC isoforms (e.g. HDAC 1, 4, and/or 6).

In another aspect, the present invention provides a method of increasing cellular re-expression of p21. The method includes contacting a cell with an HDAC inhibitor of the present invention. The amount of HDAC inhibitor is effective to increase the cellular re-expression of p21 (i.e. an effective amount). In some embodiments, the cell is a cancer cell.

In another aspect, the present invention provides a method of decreasing cellular proliferation. The method includes contacting a cell, or plurality of cells, with an HDAC inhibitor of the present invention. The amount of HDAC inhibitor is effective to decrease cellular proliferation relative to the absence of HDAC inhibitor (i.e. an effective amount). In some embodiments the cell is a cancer cell (thereby decreasing or preventing cancer progression) and/or the plurality of cells is a tumor (thereby decreasing tumor size).

In another aspect, the present invention provides a method of decreasing cellular histone acetylation. The method includes contacting the cell with an effective amount of an HDAC inhibitor of the present invention.

In another aspect, the present invention provides a method of inducing cell cycle arrest in a cell. The method includes contacting the cell with an effective amount of an HDAC inhibitor of the present invention.

In some embodiments, the compounds of the present invention are capable of inhibiting HDAC catalytic activity while avoiding excessive cytotoxic effects to non-cancerous cells (e.g. the cytotoxic effects of MS-275). Thus, in some embodiments, the compounds of the present invention are less cytotoxic than MS-275 toward non-cancerous cells.

In another aspect, the present invention provides a method of treating cancer in a subject in need thereof. The method includes administering to the subject a therapeutically effective amount of an HDAC inhibitor. A “therapeutically effective amount,” as used herein, is an amount effective to treat cancer. In some embodiments, the HDAC inhibitor forms part of a pharmaceutical formulation include the HDAC inhibitor itself and a pharmaceutically acceptable excipient. In some embodiments the subject is a mammal. In other embodiments, the subject is a human.

As used herein, the term “cancer” refers to all types of cancer, neoplasm, or malignant tumors found in mammals, including leukemia, carcinomas and sarcomas. The term “cancer” includes breast cancer, lung cancer, melanoma, colorectal cancer, bladder cancer, ovarian cancer, prostate cancer, renal cancer, squamous cell cancer, glioblastoma, pancreatic cancer, Kaposi's sarcoma, multiple myeloma, and leukemia (e.g. myeloid, chronic myeloid, acute lymphoblastic, chronic lymphoblastic, Hodgkins, and other leukemias and hematological cancers).

The term “leukemia” refers broadly to progressive, malignant diseases of the blood-forming organs and is generally characterized by a distorted proliferation and development of leukocytes and their precursors in the blood and bone marrow, and includes acute nonlymphocytic leukemia, chronic lymphocytic leukemia, acute granulocytic leukemia, chronic granulocytic leukemia, acute promyelocytic leukemia, adult T-cell leukemia, a leukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

### V. Assays

Using assays generally known in the art, compounds of the present invention may be assayed for their ability to inhibit HDAC catalytic activity, induce cell cycle arrest, increase histone acetylation, selectively inhibit the catalytic activity of specific HDAC isoforms, inhibit cellular proliferation, avoid excessive cytotoxic effects, and/or assess functionality in vivo.

A. Enzyme Inhibition Assays

All compounds produced using the synthetic routes described above can be evaluated for their ability to inhibit isolated HDAC using a commercially available assay (Fluor de Lys™ Assay System, Biomol International, LP, Plymouth Meeting, Pa.), employing TSA, SAHA and MS-275 as positive controls. This is a facile and rapid 96-well plate-based assay that is suitable for use as a high throughput screen. The reaction mixture contains a HeLa cell nuclear extract and a commercial substrate containing acetylated lysine side chains. The substrate and extract are incubated in the presence of the appropriate concentration of the inhibitor. Deacetylation of the substrate followed by mixing with the provided developer generates a fluorophore. Comparison of inhibited vs. control relative fluorescence using a standard plate reader is employed to determine percent HDAC activity remaining. All determinations can be carried out in triplicate, and reported values can be the average of these determinations. Using this assay procedure, it is possible to determine Ki values for each compound using standard Dixon plots (Wu, Y. Q. and Woster, P. M., Bioorg Med. Chem. 1993, 1, 349-360).

Additional in vitro HDAC assays can be used to screen promising compounds for selective inhibition of HDAC isoforms. HDACs 1, 3, 6 and 8 can be produced using previously described expression techniques. HDACs 1 and 3 can be isolated as fusion proteins with glutathione-S-transferase (GST) according to the method of Hu (Hu et al., J. Pharmacol. Exp. Ther. 2003, 307, 720-728). Pure HDAC 6 can be isolated from HeLa cell lysates using nickel affinity chromatography with Ni-NTA agarose columns (Qiagen, Valencia, Calif.), followed by further purification using anion exchange chromatography (Q2 column, BioRad, Hercules, Calif.) as described by Glaser (Glaser et al., Biochem Biophys Res Commun. 2004, 325, 683-690). Purified his-tagged HDAC8 can be expressed in E. coli and isolated by nickel-NTA chromatography using the method of Hu (Hu et al., J. Biol. Chem. 2000, 275, 15254-15264).

Assays for HDAC isoform selectivity can be conducted in the Biomol Fluor-de-Lys assay (Biomol, Plymouth Meeting, Pa.), substituting the appropriate amount of recombinant HDAC for the HeLa cell extract provided in the kit. Expression constructs for all known HDACs have been generated (Taunton et al., Science 1996, 272, 408-411; Yang et al., Proc. Natl. Acad. Sci. USA. 1996, 93, 12845-12850; Yang et al., J. Biol. Chem. 1997, 272, 28001-28007; Grozinger et al., Proc. Natl. Acad. Sci. USA. 96, 4868-4873; Hu et al., J. Biol. Chem. 2000, 275, 15254-15264; Kao et al., Genes Dev. 2000, 14, 55-66; Van den Wyngaert et al., FEBS Lett. 2000, 478, 77-83; Zhou et al., Proc Natl Acad Sci USA 2001, 98, 10572-10577; Gao et al., J. Biol. Chem. 2002, 277, 25748-25755), and the inhibitor selectivity of the query compound can be assessed in these preparations as well, if such studies are warranted.

B. Structural Studies

The crystal structure of HDAC 8 has already been solved (Hu et al., J. Biol. Chem. 2000, 275, 15254-15264), and can provide the structural framework for further design refinements. Once the backbone shift assignments for the protein are completed, the NMR chemical shift perturbation experiments of inhibitors added to labeled protein sample can be used to map residues that have altered chemical environments onto the crystal structure. Triple labeled (²H, ¹³C, ¹⁵N) protein can be prepared and utilized in these studies. TROSY versions of the standard backbone triple resonance experiments (HNCACB and CBCA(CO)NH) can be employed to identify and sequentially assign backbone resonances (Wittekind et al., J. Magn. Res. B., 1993, 101, 201-205). HNCACB spectra provide Ca and Cb chemical shifts for the amino acid of interest, as well as from the residue immediately N-terminal in the protein's sequence. CBCA(CO)NH spectra are used to verify chemical shifts of the N-terminal residue. Nitrogen filtered heteronuclear Nuclear Overhauser Effect (NOE) data can be used to confirrn sequential backbone assignments based on amide to amide NOEs (Noggle, J. H. and Shirmer, R. E., The Nuclear Overhauser Effect Chemical Applications, 1971, New York: Academic Press. 1-259). When combined, these NMR data allows sequential assignments to be made for the majority of the amide backbone resonances in the protein. Once backbone amide assignments have been made, amide NMR assignments are used to further refine HDAC inhibitors binding mechanisms. A simple 2D ¹H, ¹⁵N TOCSY-HSQC experiment provide the means to monitor chemical shift perturbations of amide resonances during the addition of inhibitor to the protein. Inhibitor can be directly added to the NMR tube containing labeled protein.

C. Cellular Assays

The well characterized ML-1 human myelocytic leukemia cell line was used to obtain the results presented below in the Examples section. This line has a well characterized response to HDAC inhibitors and is amenable for use in rapid in vitro screening of compounds to evaluate growth inhibitory effects. In addition to the ML-1 cells, the well characterized HCT 116 human colon adenocarcinoma may be used. This model represents an important human solid tumor and has been useful in identifying the effectiveness of other classes of HDAC inhibitors (Hinnebusch et al., J Nutr. 2002, 132, 1012-1017; Tan et al., Int J Cancer 2002, 98, 523-531; McBain et al., Biochem Pharmacol. 1997, 53, 1357-1368; Suzuki et al., J. Med. Chem. 2005, 48, 1019-1032, Qian et al., Cancer Res. 2004, 64, 6626-6634; Sandor et al., Clin Cancer Res. 2002, 8, 718-728; Roy et al., Cell Death Differ. 2005, 12, 482-491). The HCT 116 model has the added advantage of having several isogenic lines available in which single genes have been “knocked out” by homologous recombination. The use of appropriate cell lines (including the p53 −/− and p21^{waf1} −/− lines) are described in Qian et al., Cancer Res. 2004, 64, 6626-6634; Sandor et al., Clin Cancer Res. 2002, 8, 718-728; Roy et al., Cell Death Differ. 2005, 12, 482-491; Waldman et al., Cancer Res. 1995, 55, 5187-5190; Bunz et al., Science 1998, 282, 1497-1501; Bunz et al., J. Clin. Invest. 1999, 104, 263-269. These isogenic lines allow not only the evaluation of the effects of HDAC inhibition and growth inhibitory effects, but also the direct determination of p53 dependence and the role of p21 re-expression in determining cellular response. Additionally, the HCT 116 cell lines provides relevant in vivo human solid tumor xenograft models with which the in vivo activity of candidate PAHI inhibitors can be examined.

The MTT (3-[4,5-dimethylthiazol-2-yl]2,5-diphenyl tetrazolium bromide) reduction assay for growth inhibition studies may also be employed. The standard 96 well plate assay allows multiple compounds tested in duplicate over a 4 log range of concentrations. HCT 116 cells are seeded at a density of 5×104 cells/ml and ML-1 cells are seeded at 2×104/ml and are treated for a period of 96 hr prior to dye reduction and plate reading. The growth inhibitory effect of compounds selected form the MTT results may be verified by trypan blue exclusion cell counts.

Advantages of the increased targeting of the present compounds may include increased anti-tumor affects, while decreasing toxicity to normal cells. The combination of HDAC inhibitors with other agents may increase antitumor effects of the HDAC inhibitors.

For example, HDAC inhibitors in combination with DNA-demethylating agents has been shown to be clinically effective. Thus, the compounds of the present invention may be tested with demethylating agents for synergy in cytotoxicity and gene re-expression. For cytotoxicity, the 96 well MTT assay described above may be utilized, with low dose 5-aza-2′-deoxycytidine (DAC) at 100 nm (Cameron et al., Nature Genet. 1999, 21, 103-107; Cameron et al., Blood 1999, 94, 2445-2451) prior to (24 hours) treatment with effective doses of tested PAHI's. HCT 116 cells are seeded at a density of 5×10⁴ cells/ml and ML-1 cells are seeded at 2×10⁴/ml and are treated for a period of 96 hr prior to dye reduction and plate reading.

As previously examined classes of HDAC inhibitors have demonstrated the ability to induce cell cycle arrest and the induction of this arrest is directly associated with the antiproliferative activity of those agents, the ability of the compounds of the present invention to disrupt the cell cycle and induce apoptosis may be examined. Cell cycle arrest and apoptosis may be evaluated by flow cytometry of PI/BUdR stained ML-1 and HCT116.

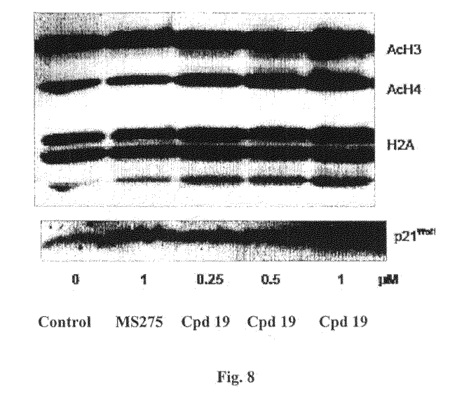

The ability of the PAHIs to alter histone acetylation in a dose and time dependent manner may be assayed in ML-1 and HCT 116 cells using the same techniques that were used to produce the results in FIG. 8. Briefly, after treatment, cells are washed in 2 ml HBSS and disrupted by 1 mL ice-cold lysis buffer A (10 mM Tris pH 7.6, 5 mM butyric acid, 1% Triton X-100, 1 mM MgCl₂, and 1 mM PMSF). Nuclei are collected by centrifugation at 14,000 rpm for 15 min. The pellets are resuspended with 250 μL ice-cold lysis buffer B (10 mM Tris pH 7.6, 0.25 M Sucrose, 3 mM CaCl₂, and 5 mM butyric acid). Sulfuric acid is added to a concentration of 0.4 N and samples are incubated at 4° C. for overnight. Debris is pelleted by centrifugation, and the supernatant collected. Histones are precipitated by addition of 10 vol of acetone and incubated at −20° C. overnight. Pellets are collected by centrifugation, briefly dried under vacuum, and resuspended in ddH₂O. Proteins (10 μg each lane) are separated by 15% SDS PAGE and visualized by Western blot analysis using primary antibodies against acetyl-histone H3 (diluted 1:1000), acetyl-histone H4 (diluted 1:500), and histone-1-12A (diluted 1:1000) from Upstate Biotechnologies. The immunoreactive proteins are quantified using the Odyssey Infrared Imaging System (Li-Cor Bioscience).

PAHIs assayed for their ability to increase the expression of the CDKI, p21^{waf1} in treated ML-1 leukemia and HCT 116 colon cancer cells alone and in combination with DNA-demethylating agent, DAC. The expression of p21^{waf1} may be determined by Western analysis similar to the methods used for determination of the acetylated histones with the exception that 30 pg of protein are used and the p21 primary antibody (BD Pharmigen) is diluted 1:500.

To examine area of active chromatin and inactive chromatin at specific promoters, Chromatin Immunopercipitation may be performed in the region surrounding the transcription start site of the p21 gene, before and after treatment with PAHIs. MS275 (300 nM to 1 micromolar) and TSA (300 nM), well characterized HDAC inhibitors, may be used as controls. The ChIP Assay Kit from Upstate Biotechnology amay be employed (Fahrner et al., Cancer Res. 2002, 62, 7213-7218), following the manufacturer's protocol with some modifications. Proteins are cross-linked to DNA by addition of formaldehyde directly to the culture medium to a final concentration of 1% for 10 min at room temperature. The cross-linking reaction is quenched by adding glycine to a final concentration of 0.125 M for 5 min at room temperature. The medium is then removed and cells were washed with 1×PBS containing a combination of protease inhibitors (1 mM Pefabloc and 1× Complete protease inhibitor mixture; Roche Molecular Biochemicals). The PBS is removed and 0.2× trypsin added to the cells. After a 5-min incubation at 37° C., ice-cold 1×PBS containing 10% FBS is added to stop trypsinization. The cells are scraped off the culture flask, pelleted, and washed twice with 1×PBS plus protease inhibitors as above. For each ChIP assay 10⁶ cells are used. The sonicated samples are precleared with 80 μl of salmon sperm DNA/Protein A and Protein G agarose beads (3:1 ratio of Protein A to Protein G; Upstate Biotechnology) for 1 h at 4° C. with agitation. The soluble chromatin fraction is collected and 5 μl of specific antibodies. Anti-acetyl-Histone H3 (Lys 9 and Lys 14) is primarily examined for direct HDAC inhibition, and anti-dimethyl-Histone H3 (Lys 4), anti-dimethyl-Histone H3 (Lys 9) are also examined as loss of Histone acetylation is associated with changes in histone methylation and correlates with chromatin changes leading to gene silencing. For these assays, antibodies to histone modification (or no antibody added control) are incubated overnight with rotation (all antibodies from Upstate Biotechnology). Immune complexes are collected with 60 pl of the 3:1 salmon sperm DNA/Protein A and Protein G agarose beads. The beads are washed as recommended and transferred to a new tube before each wash. After elution, the cross-links are reversed, and the samples digested with proteinase K. DNA was recovered by phenol extraction, ethanol precipitated, and resuspended in 1×10 mM Tris (pH 8)-1 mM EDTA buffer.

Primer sets for PCR may be designed to amplify overlapping fragments of 200 by surrounding the promoter of p21 and other identified induced genes including the p15 locus in ML-1 (Herman et al., Cancer Res. 1996, 56, 722-727) and GATA-4 and GATA5 (Akiyama et al., Mol Cell Biol. 2003, 23, 8429-8439) and SFRP-1 SFRP-2 SFPRP-4, SFRP-5 and SEZ6L (Suzuki et al., Nat Genet. 2002, 31, 141-149) in HCT116. One primer set for GAPDH designed to amplify a 128-bp fragment of the genomic sequence may serve as an internal control. Primers may be purchased from Invitrogen or IDT. PCR reactions may be performed with JumpStart REDTaq DNA Polymerase (Sigma) in a total volume of 25 μl, using 1-2 μl of either immunoprecipitated (bound) DNA, a 1:10 dilution of non-immunoprecipitated (input) DNA, or a no-antibody control. Reactions may be optimized with input DNA to ensure that PCR products for both p21 and other genes and GAPDH are in the linear range of amplification. Where gel based analysis is performed, ten μl of PCR product are size fractionated by PAGE and quantified using Kodak Digital Science ID Image Analysis software. Enrichment is calculated by taking the ratio between the net intensity of the PCR product from each primer set and the net intensity of the GAPDH PCR product for the bound sample and dividing this by the same ratio calculated for the input sample. Values for enrichment are calculated as the average from at least two independent ChIP experiments and multiple independent PCR analyses of each.

D. Specificity Assays

Histone deacetylases are members of a deacetylase superfamily broadly divided into 3 groups, Class I, Class II, and the Class III SIRTs (de Ruijter et al., Biochem. J. 2003, 370, 737-749; Gray, S. G. and Ekstrom, T., Exp Cell Res. 2001, 262, 75-83). To test the relative specificity of the inhibitors, treated cells are evaluated for changes in levels of acetylated α-tubulin. The ratio of increased H3/H4 acetylation versus increased atubulin acetylation provides specificity information. The methods used to measure acetylated α-tubulin are similar to those detailed above for H3/H4 with the exception that anti-α-tubulin (Sigma) is used as the primary antibody.

More detailed in vitro HDAC assays can be used to assay PAHIs for selective inhibition. Isotype specific immunopercipitation for the main class I HDAC is accomplished by specific antibodies to HDAC 1, 2, and 3. Recombinant HDAC8 is also available, which can be used directly. Immunoprecipitation of HDAC isoforms may also be used. Immunoprecipitated or recombinant HDAC may be divided into four reactions in the absence or presence of an increasing concentration of selected PAHIs. To each reaction the Fluor De Lys acetyl-lysine peptide substrate (Biomol, Inc.) is added and incubated at 30° C. for 10 minutes. The released fluorescent product generated from histone deacetylation is quantified using a fluorimeter. In this way, the compound is screened based on their ability to inhibit deacetylation by specific HDACs. Mammalian expression constructs for all known HDACs have been generated (Taunton et al., Science 1996, 272, 408-411; Yang et al., Proc. Natl. Acad. Sci. USA. 1996, 93, 12845-12850; Yang et al., J. Biol. Chem. 1997, 272, 28001-28007; Hu et al., J. Biol. Chem. 2000, 275, 15254-15264; Van den Wyngaert et al., FEBS Lett. 2000, 478, 77-83; Gao et al., J. Biol. Chem. 2002, 277, 25748-25755; Grozinger et al., Proc. Natl. Acad. Sci. USA. 96, 4868-4873; Kao et al., Genes Dev. 2000, 14, 55-66; Zhou et al., Proc Natl Acad Sci USA 2001, 98, 10572-10577).

E. In Vivo Assays

To assess the in vivo effects of the compounds of the present invention, mice models may be used (e.g. eight week old BALB/c nu/nu athymic mice weighing between 25-30 g). The animals are typically maintained 5 to a cage under sterile conditions (refer to Section F for details on animal care).

To determine the appropriate doses small scale LD50 determination experiments are performed prior to initiation of the tumor treatments. Five dosing rates administered IP is examined in 3 animals each. The highest dose allowing 100% survival is used as the high dose and 50% of the high dose is used as the second dose. Dosing may be administered by IP injection.

Tumor cells from log phase culture are implanted into mice by inoculation of 107 tumor cells into the subscapular region. Once the tumors have reached 100-200 mm³ in volume, the animals are randomized for treatment. IP injections of the appropriate concentrations in a volume of 0.2 mL are then started.

Generally, fifteen animals for each treatment group are used. This allows the sacrifice of 5 animals from each treatment group for study of the immediate effects of treatment. The five animals are sacrificed 24 hours after the last injection. From these animals: 1) tumor and normal tissues (lung, liver, kidney) are collected for determination of polyamine and analogue pools, SSAT, ODC, AdoMetDC, and SMO/PAOhl activities, changes in histone acetylation and p21 expression; 2) gross organ or tissue toxicity is assessed.

The remaining ten animals in each of the treatment groups are followed for the determination of antitumor drug effects using time to progression-Kaplan-Meier analysis. Progression is defined as a tumor volume 4 times the volume of the tumor at initiation of treatment. The logrank test is used to determine the statistical significance of any tumor response to the test agents. Differences may be considered statistically significant if p<0.05. Tumor volume may be estimated weekly.

### VI. Pharmaceutical Compositions and Administration

In another aspect, the present invention provides a pharmaceutical composition including a HDAC inhibitor in admixture with a pharmaceutically acceptable excipient. One of skill in the art will recognize that the pharmaceutical compositions include the pharmaceutically acceptable salts of the HDAC inhibitors described above.

In therapeutic and/or diagnostic applications, the compounds of the invention can be formulated for a variety of modes of administration, including systemic and topical or localized administration. Techniques and formulations generally may be found in Remington: The Science and Practice of Pharmacy (20th ed.) Lippincott, Williams & Wilkins (2000).

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A most preferable dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the invention may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a subject (e.g. patient) to be treated.

For nasal or inhalation delivery, the agents of the invention may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Depending upon the particular condition, or disease state, to be treated or prevented, additional therapeutic agents, which are normally administered to treat or prevent that condition, may be administered together with the inhibitors of this invention. For example, chemotherapeutic agents or other anti-proliferative agents may be combined with the inhibitors of this invention to treat proliferative diseases and cancer. Examples of known chemotherapeutic agents include, but are not limited to, adriamycin, dexamethasone, vincristine, cyclophosphamide, fluorouracil, topotecan, taxol, interferons, and platinum derivatives.

Other examples of agents the inhibitors of this invention may also be combined with include, without limitation, anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents for treating diabetes such as insulin, insulin analogues, alpha glucosidase inhibitors, biguanides, and insulin sensitizers; and agents for treating immunodeficiency disorders such as gamma globulin.

These additional agents may be administered separately, as part of a multiple dosage regimen, from the inhibitor-containing composition. Alternatively, these agents may be part of a single dosage form, mixed together with the inhibitor in a single composition.

The present invention is not to be limited in scope by the exemplified embodiments, which are intended as illustrations of single aspects of the invention. Indeed, various modifications of the invention in addition to those described herein will become apparent to those having skill in the art from the foregoing description. Such modifications are intended to fall within the scope of the invention. Moreover, any one or more features of any embodiment of the invention may be combined with any one or more other features of any other embodiment of the invention, without departing from the scope of the invention. For example, the HDAC inhibitors described in the Histone Deacetylase Inhibitors section are equally applicable to the methods of treatment and methods of inhibiting kinases described herein. References cited throughout this application are examples of the level of skill in the art and are hereby incorporated by reference herein in their entirety for all purposes, whether previously specifically incorporated or not.

### VII. Examples

The following examples are offered to illustrate, but not to limit the claimed invention.

All reagents were purchased from Aldrich Chemical Co. (Milwaukee, Wis.), Sigma Chemical Co. or Acros Chemical (Chicago, Ill.) and were used without further purification except as noted below. Pyridine was dried by passing it through an aluminum oxide column and then stored over KOH. Triethylamine was distilled from potassium hydroxide and stored in a nitrogen atmosphere. Methanol was distilled from magnesium and iodine under a nitrogen atmosphere and stored over molecular sieves. Methylene chloride was distilled from phosphorus pentoxide and chloroform was distilled from calcium sulfate. Tetrahydrofuran was purified by distillation from sodium and benzophenone. Dimethyl formamide was dried by distillation from anhydrous calcium sulfate and was stored under nitrogen. Preparative scale chromatographic procedures were carried out using E. Merck silica gel 60, 230-440 mesh. Thin layer chromatography was conducted on Merck precoated silica gel 60 F-254. Ion exchange chromatography was conducted on Dowex IX8-200 anion exchange resin. Compounds 15 and 16 used in Schemes 2-4 were synthesized as previously described.²

All ¹H- and ¹³C-NMR spectra were recorded on a General Electric QE-300 or a Varian Mercury 400 mHz spectrometer, and all chemical shifts are reported as δ values referenced to TMS or DSS. Infrared spectra were recorded on a Nicolet 5DXB FT-IR spectrophotometer and are referenced to polystyrene. In all cases, ¹H-NMR, ¹³C-NMR and IR spectra were consistent with assigned structures. Melting points were recorded on a Thomas Hoover Capillary melting point apparatus and are uncorrected. Mass spectra were recorded on a Kratos MS 80 RFA (EI and CI) or Kratos MS 50 TC (FAB) mass spectrometers. Microanalyses were performed by Galbraith Laboratories, Knoxyille, Tenn., and were within 0.4% of calculated values.

ML-1 cells were maintained in RPMI medium supplemented with 10% fetal calf serum, 0.1 mg/mL gentamicin, and 2 mM L-glutamine. 3×10⁵ cells/ml were treated with 1 (Wako Pure Chemicals, Richmond, Va.), 2 (Mitsui Pharmaceuticals, Chiba, Japan) and the desired test compound for the concentration and time indicated in the figure legend.

Histones were prepared by a modification of a previously described method. Cells were washed in 2 mL of HBSS and disrupted by 1 mL of ice-cold lysis buffer A (10 mM Tris pH 7.6, 5 mM butyric acid, 1% Triton X-100, 1 mM MgCl₂, and 1 mM PMSF). Nuclei were collected by centrifugation at 14 000 rpm for 15 min. The pellet was resuspended once with 250 μL of ice-cold lysis buffer B (10 mM Tris pH 7.6, 0.25 M Sucrose, 3 mM CaCl₂, and 5 mM butyric acid). Sulfuric acid was added to a concentration of 0.4 N, and the tubes were incubated at 4° C. for overnight. Debris was pelleted by centrifugation, and the supernatant was collected. Histones were precipitated by addition of 10 vol of acetone and incubated at −20° C. overnight. Pellets were collected by centrifugation, briefly dried under vacuum, and resuspended in ddH₂O.

A. Chemical Synthesis and Compound Characterization

N-[4-Azido)butyl]phthalimide (22). A 1.0 g portion of N-[4-(bromo)butyl]phthalimide 21 (0.0035 mol) was dissolved in 10 mL of DMF, and to this solution was added 0.290 g (0.0044 mol) of sodium azide. The reaction was then allowed to stir for 5 h under nitrogen, after which the reaction mixture was concentrated in vacuo to yield a white semisolid. The semisolid was dissolved in water and extracted with three 50 mL portions of ethyl acetate, the combined organic layers were dried over anhydrous magnesium sulfate and filtered, and the solvent was removed to afford 22 (0.760 g, 88%) as a white amorphous powder. This preparation was used in the next reaction without further purification. ¹H NMR (400 MHz CDCl₃) δ 1.6-1.68 (m, 2H), 1.74-1.82 (m, 2H), 3.3 (t, J=7.2 Hz, 2H), 3.7 (t, J=7.2 Hz, 2H), 7.71-7.73 (m, 2H), 7.83-7.86 (m, 2H). ¹³C NMR (400 MHz CDCl₃) δ 26.1, 28.6, 44.02, 53.6, 127.4, 128.0, 132.34, 134.12, 168.52, 171.0.

N-[4-(Amino)butyl]phthalimide (23). Compound 22 (0.760 g, 0.0031 mol) was dissolved in 50 mL of ethanol along with 0.100 g of 10% Pd/C, and the suspension was hydrogenated at 25 psi for 12 h. The reaction mixture was then filtered, and the filtrate was concentrated in vacuo to yield 23 as an amorphous white solid (0.620 g, 92%) that was of sufficient purity to use in the next reaction. ¹H NMR (400 MHz CD₃OD) δ 1.6-1.8 (m, 4H), 2.9 (t, J=7.2 Hz, 2H), 3.7 (t, J=7.6 Hz, 2H), 7.9 (m, 4H). ¹³C NMR (400 MHz CDCl₃) δ 26.1, 33.8, 44.0, 46.4, 127.4, 128.0, 132.23, 134.12, 168.25, 171.0

N-{4-[(N,N-Dimethylamino)benzyl]butyl}phthalimide (24c). A 0.589 g portion of 23 (0.0027 mol) was dissolved in 20 mL of dichloroethane, and to this solution N,N-dimethylaminobenzaldehyde (0.477 g, 0.0032 mol) was added along with 0.186 g (0.0031 mol) of acetic acid. The reaction was allowed to stir at room temperature for 20 min. after which time sodium cyanoborohydride (0.220 g, 0.0035 mol) was dissolved in 3 mL of methanol and added to the reaction, which was then allowed to stir for an additional 12 h. The reaction mixture was concentrated on a rotary evaporator, and the resulting yellow oil was dissolved in water and extracted with three 50 mL portions of chloroform. The organic layers were combined, washed with brine, and then dried over anhydrous magnesium sulfate. The solution was filtered and concentrated in vacuo to give crude 24a as a cloudy yellow oil. The crude compound was purified using column chromatography (hexane:ethyl acetate 1:3 followed by ethyl acetate:methanol 2:1), to yield 25a as a clear yellow oil (0.810 g, 85.4%). ¹H NMR (400 MHz CDCl₃) δ 1.55 (q, J=7.2 Hz, 2H), 1.72 (q, J=7.2 Hz, 2H), 2.65 (t, J=7.6 2H), 2.92 (s, 6H), 3.69 (s, 2H), 3.71 (t, J=7.2 Hz, 2H), 6.7 (d, 2H), 7.1 (d, 2H), 7.68-7.71 (m, 2H), 7.81-7.83 (m, 2H). ¹³C NMR (400 MHz, CDCl₃) δ 24.35, 25.35, 40.9, 50.06, 112.85, 127.4, 128.0, 128.6, 132.34, 134.12, 150.0, 168.52, 169.0.

N-{4-[4-(Methyl)benzylamino]butyl}phthalimide (24b). Compound 24b was synthesized from 24 and 4-methylbenzaldehyde according to the procedure used to synthesize 24c in 73.2% yield. ¹H NMR (400 MHz, CDCl₃) δ 1.53 (q, J=7.2 Hz, 2H), 1.70 (q, J=7.2 Hz, 2H), 2.31 (s, 3H), 2.62 (t, J=7.6 2H), 6H), 3.68 (s, 2H), 3.70 (t, J=7.2 Hz, 2H), 6.7 (d, 2H), 7.1 (d, 2H), 7.68-7.71 (m, 2H), 7.81-7.83 (m, 2H). ¹³C NMR (400 MHz CDCl₃) δ 25.52, 26.19, 37.85, 46.02, 53.65, 60.61, 123.41, 127.36, 127.95, 129.33, 132.34, 134.12, 136.92, 168.59.

N-{4-[2-(Phenyl)benzylamino]butyl}phthalimide (24a). Compound 24a was synthesized from 23 and 2-(phenyl)-benzaldehyde according to the procedure used to synthesize 24c in 75% yield. ¹H NMR (400 MHz CDCl₃) δ 1.42 (q, J=7.6 Hz, 2H), 1.59 (q, J=7.2 Hz, 2H), 2.54 (t, J=7.2 Hz, 2H), 3.59 (t, J=7.2 Hz, 2H), 3.84 (s, 2H), 7.19-7.45 (complex m, 9H), 7.77-7.81 (m, 4H). ¹³C NMR (400 MHz CDCl₃) δ 25.52, 26.18, 37.8, 47.15, 48.07, 123.38, 126.98, 127.34, 127.86, 128.461, 129.37, 139.06, 132.35, 134.10, 141.0, 146.9, 168.50.

1-N-{4-[(N,N-Dimethyl)amino benzyl}-1-N-[(tert-butyloxy)carbonyl]-4-phthalimidobutylamine (25c). A 0.800 g portion of 24c (0.0023 mol) was dissolved in 20 mL of dichloromethane, and the reaction mixture was cooled to 0° C. To this mixture was added an aqueous solution of sodium bicarbonate (0.220 g, 0.0027 mol) and sodium chloride (0.160 g, 0.0027 mol), and the reaction was allowed to stir at 0° C. for 30 min. Di-tert-butyl dicarbonate (0.596 g, 0.0027 mol) was dissolved in 5 mL of dichloromethane, and the solution was slowly added to the reaction. The mixture was allowed to stir at 0° C. for an additional 10 min and warmed to room temperature, followed by reflux for 12 h. The reaction mixture was cooled and extracted with three 25 mL portions of dichloromethane. The combined organic layers were washed with 50 mL of saturated sodium bicarbonate and 50 mL of saturated sodium chloride solution and then dried over anhydrous magnesium sulfate. The mixture was then filtered, and the solvent was removed in vacuo to yield crude 25c. The crude compound was purified on a silica gel column eluted with hexane:ethyl acetate (4:3) and then ethyl acetate (100%), to yield compound 25c as a clear yellow oil (0.950 g, 91.5%). ¹H NMR (400 MHz CDCl₃) δ 1.4 (s, 9H), 1.51-1.61 (broad m, 4H), 2.89 (s, 6H), 3.1-3.18 (broad m, 2H), 3.65 (t, 2H), 4.3 (s, 2H), 6.65 (d, J=6.4 Hz, 2H), 7.09 (broad s, 2H), 7.68-7.71 (m, 2H), 7.81-7.83 (m, 2H). ¹³C NMR (400 MHz CDCl₃) δ 25.52, 26.19, 28.66, 37.85, 46.02, 53.65, 60.61, 79.82 123.41, 127.36, 127.95, 129.33, 132.34, 134.12, 136.92, 168.59. IR (cm⁻¹) 3410.2, 2942.8, 1651.8, 1555.8, 1532.2, 1460.12, 1105.32.

1-N-[4-(Methyl)benzyl]-1-N-[(tert-butyloxy)carbonyl]-4-phthalimidobutylamine (25b). Compound 25b was synthesized from 24b according to the procedure used to synthesize 24c in 92% yield. ¹H NMR (400 MHz CDCl₃) δ 1.44 (s, 9H), 1.53-1.63 (m, 4H), 2.30 (s, 3H), 3.14-3.22 (broad d, 2H), 3.66 (t, J=7.2 Hz, 2H), 4.36 (s, 2H), 7.09 (s, 2H), 7.71 (m, 2H), 7.84 (m, 2H). ¹³C NMR (400 MHz CDCl₃) δ 24.64, 26.19, 28.71, 39.03, 40.92, 51.77, 60.61, 79.82 112.85, 123.41, 127.36, 127.95, 129.33, 132.34, 134.12, 136.92, 150.66, 156.17. IR (cm⁻¹) 2962.5, 2942.8, 1768.7, 1710.3, 1684.3, 1619.4, 1487.2, 1365.4, 1301.4.

1-N-[2-(Phenyl)benzyl]-1-N-[(tert-butyloxy)carbonyl]-4-phthalimidobutylamine (25a). Compound 25a was synthesized from 24a according to the procedure used to synthesize 24c in 90.2% yield. ¹H NMR (400 MHz CDCl₃) δ 1.38-1.42 (m, 1H), 1.52 (m, 2H), 2.96 (broad s 1H), 3.09 (broad s 1H), 3.56 (broad s, 2H), 4.34 (s, 1H), 4.43 (s, 1H), 7.18 (d, J=7.6 Hz, ¹H), 7.21-7.4 (m, 8H), 7.70-7.77 (m, 2H), 7.8-7.83 (m, 2H). ¹³C NMR (400 MHz CDCl₃) δ 25.52, 26.18, 27.63, 28.62, 37.8, 47.15, 48.07, 79.83, 85.4123.38, 126.98, 127.34, 127.86, 128.461, 129.37, 139.06, 132.35, 134.10, 141.0, 146.9, 168.50.

1-N-{4-[(N,N-Dim ethyl)amino]benzyl}-1-N-[(tert-butyloxy)carbonyl]-4-aminobutylamine (26c). A 0.90 g (0.0019 mol) portion of 25c was dissolved in 10 mL of methanol, and 0.191 g (0.0059 mol) of hydrazine was added dropwise with stirring. The reaction was allowed to reflux under nitrogen for 12 h, and then the reaction mixture was concentrated in vacuo to yield a white semisolid. This solid was dissolved in 50 mL of 4.0 N ammonium hydroxide and extracted with three 50 mL portions of chloroform. The combined organic layers were dried over magnesium sulfate, filtered and concentrated in vacuo, to yield 26e (0.560 g, 88.3%) as a white amorphous solid. ¹H NMR (400 MHz CDCl₃) δ 1.37 (broad m, 2H), 1.45 (s, 9H), 1.47 (m, 2H), 2.6 (t, J=7.2 Hz, 2H), 2.92 (s, 6H), 3.09 (broad m, 2H), 4.3 (s, 2H), 6.6 (d, 2H), 7.1 (m, 2H). ¹³C NMR (400 MHz CDCl₃) δ 26.19, 28.66, 37.85, 46.02, 53.65, 60.61, 79.82 123.41, 112.85, 127.36, 127.95, 168.59. IR (cm⁻¹) 3365.8, 2974.8, 2929.7, 1689.8, 1570.5, 1467.0, 1310.1, 1168.6.

1-N-[4-(Methyl)benzyl]-1-N-[(tent-butyloxy)carbonyl]-4-aminobutylamine (26b). Compound 26b was synthesized from 25b according to the procedure used to synthesize 26c in 82.8% yield. ¹H NMR (400 MHz CDCl₃) δ 1.38 (m, 2H), 1.44-1.48 (m, 11H), 2.32 (s, 3H), 2.66 (t, J=6.8 Hz, 2H), 3.11-3.19 (broad m, 2H), 4.38 (s, 2H), 7.11 (s, 4H). ¹³C NMR (400 MHz CDCl₃) δ 25.39, 28.67, 31.22, 42.13, 46.36, 49.68, 50.13, 79.69, 115.28, 127.33, 127.92, 129.33, 135.67, 138.90. IR (cm⁻¹) 3360.6, 2975.8, 2929.3, 1690.8, 1514.9, 1410.6, 1365.3, 1245.0.

1-N-[2-(Phenyl)benzyl]-1-N-[(tert-butyloxy)carbonyl]-4-aminobutylamine (26a). Compound 26a was synthesized from 25a according to the procedure used to synthesize 26c in 78.6% yield. ¹H NMR (400 MHz CDCl₃) δ 1.24-1.31 (m, 4H), 1.461 (s, 9H), 2.56 (m, 2H), 2.95 (broad s, 1H), 3.08 (bs, 1H), 4.35 (s, 1H), 4.45 (s, 1H), 7.31-7.41 (m, 9H) ¹³C NMR (400 MHz CDCl₃) δ 25.32, 25.38, 28.56, 28.63, 31.08, 42.06, 46.19, 46.63, 48.05, 79.72, 126.97, 127.36, 127.85, 128.45, 129.36, 130.12, 141.06 IR (cm⁻¹) 3372.9, 2929.4, 1683.0, 1569.4, 1514.2, 1473.2, 1198.7.

5-{4-[N-(tert-Butyloxy)carbonyl]-4-[N,N-(dimethylamino)benzyl]amino}butylcarbamoylpentanoic Acid Methyl Ester (28d). A 0.500 g (0.0051 mol) portion of 26c was dissolved in 15 mL of dichloromethane, and the reaction mixture was cooled to 0° C. Two to three drops of triethylamine was then added to the solution, and the reaction was allowed to stir for 15 min. The acid chloride 27a (0.333 g, 0.0019 mol) was slowly added to the reaction mixture, which was allowed to stir at 0° C. for 15 min, then heated to room temperature and allowed to stir for an additional 8 h. The solvent was removed in vacuo, and the residue was dissolved in water and extracted with three 50 mL portions of chloroform. The organic layers were combined and washed with 50 mL of saturated sodium bicarbonate and 50 mL of saturated sodium chloride and then dried over magnesium sulfate. Filtration and removal of the solvent in vacuo then afforded crude 28d. Purification on silica gel (hexane:EtOAc 1:3) then gave pure 28d (0.580 g, 80.2%) as a clear yellow oil. ¹H NMR (CDCl₃) δ 1.2 (m, 4H), 1.4 (s, 9H), 1.5 (m, 4H), 2.11 (t, J=7.2 Hz, 2H), 2.28 (t, J=7.6 Hz, 2H), 2.93 (s, 6H), 3.22 (m, 4H), 3.6 (s, 3H), 4.3 (s, 2H), 6.6 (d, J=8.4 Hz, 2H), 7.1 (m, 2H). ¹³C NMR (400 MHz CDCl₃) δ 24.64, 25.35, 26.32, 28.71, 33.89, 34.01, 36.41, 39.30, 40.92, 45.57, 50.06, 51.77, 79.72, 112.84, 128.66, 150.06, 156.17, 172.93, 174.21 IR (cm⁻¹) 3416.2, 2942.7, 1736.2, 1651.9, 1554.6, 1522.2, 1256.8.

7-(4-[(tert-butyloxy)carbonyl]-[4-(methyl)benzyl]-amino/butylcarbamoylheptanoic Acid Methyl Ester (28e). Compound 28e was synthesized from 26b and 27b according to the procedure used to synthesize 28d in 64.8% yield. ¹H NMR (400 MHz CDCl₃) δ 1.33 (m, 4H), 1.46 (broad s, 13H), 1.62 (m, 4H), 2.14 (m, 2H), 2.29 (m, 2H), 2.39 (s, 3H), 3.22 (m, 4H), 3.66 (s, 3H), 4.37 (s, 2H), 7.12 (s, 4H). ¹³C NMR (400 MHz CDCl₃) TM 24.94, 25.55, 25.77, 26.46, 28.66, 28.99, 29.08, 34.17, 36.85, 39.23, 46.04, 49.85, 50.4, 51.66, 80.01, 127.41, −127.94, 129.36, 135.55, 137.00, 156.13, 173.30, 174.41. IR (cm⁻¹) 3607.4, 3328.7, 2936.2, 2858.4, 1738.6, 1691.8, 1652.0, 1548.6, 1509.1.

7-{4-[(tert-Butyloxy)carbonyl]-4-[N,N-(dimethylamino) benzyl]amino}butylcarbamoylheptanoic Acid Methyl Ester (28c). Compound 28c was synthesized from 26c and 27b according to the procedure used to synthesize 28d in 76.5% yield. ¹H NMR (CDCl₃) δ 1.40-1.59 (m, 13H), 1.60 (m, 8H), 2.11 (m, 2H), 2.3 (m, 2H), 2.95 (s, 6H), 3.20 (m, 4H), 3.66 (s, 3H), 4.23 (s, 2H), 6.69 (d, J=8 Hz, 2H), 7.11 (broad s, 2H). ¹³C NMR (400 MHz CDCl₃) δ 24.64, 25.35, 26.32, 28.25, 28.64, 28.71, 29.07, 33.89, 35.98, 36.88, 40.01, 41.13, 46.57, 50.07, 51.92, 80.02, 128.66, 129.50, 150.06, 156.17, 172.93, 174.21. IR (cm⁻¹) 3437.5, 2956.6, 1739.3, 1651.0, 1550.6, 1522.2, 1256.8.

5-{4-[(tert-butyloxy)carbonyl]-[4-(methyl)benzyl]-amino}butylcarbamoylpentanoic Acid Methyl Ester (28b). Compound 28b was synthesized from 26b and 27a according to the procedure used to synthesize 28d in 70.7% yield. ¹HNMR (400 MHz CDCl₃) δ 1.44 (broad s, 13H), 1.60 (m, 4H), 2.14 (m, 2H), 2.29 (m, 2H), 2.4 (s, 3H), 3.22 (m, 4H), 3.67 (s, 3H), 4.33 (s, 2H), 7.12 (s, 4H). ¹³C NMR (400 MHz CDCl₃) δ 25.77, 26.46, 28.99, 29.08, 34.17, 36.85, 39.23, 46.04, 49.85, 50.4, 51.66, 80.01, 127.41, 127.94, 129.36, 135.55, 137.00, 156.13, 173.30, 174.41. IR (cm⁻¹) 3338.4, 3318.9, 2936.2, 2858.4, 1736.2, 1690.8, 1651.9, 1548.1, 1509.1.

5-{4-[(tert-butyloxy)carbonyl]-[2-(phenyl)benzyl]-amino}butylcarbamoyl}pentanoic Acid Methyl Ester (28a). Compound 28a was synthesized from 26a and 27a according to the procedure used to synthesize 28d in 62.4% yield. ¹H NMR (400 MHz CDCl₃) δ 1.23-1.48 (m, 13H), 1.62-1.71 (m, 4H), 2.15 (m, 2H), 2.31-2.38 (m, 2H), 2.96 (s, 1H), 3.08 (m, 4H), 3.64 (s, 3H), 4.35 (s, 1H), 4.43 (s, 1H), 6.26 (s, ¹H), 7.20-7.42 (m, 9H). ¹³C NMR (400 MHz CDCl₃) δ 24.86, 28.33, 36.502, 39.35, 46.12, 47.14, 48.15, 51.73, 51.78, 60.60, 79.97, 126.76, 126.92, 127.41, 127.59, 127.85, 128.49, 129.31, 130.15, 172.93, 173.48, 174.13, 175.72. IR (cm⁻¹) 3304.9, 2921.0, 1722.3, 1671.4, 1651.9, 1551.6, 1158.9.

5-{4-[N-(tert-Butyloxy)carbonyl]-4-[N,N-(dimethylamino)benzyl]amino}butylcarbamoylpentanoic Acid (29d). A 0.500 g (0.0011 mol) portion of 28d was dissolved in 6 mL of tetrahydrofuran:water (4:2) and cooled to 0° C., and 6 mL of 1.0 N LiOH was added to the mixture by dropwise addition. The solution was warmed to room temperature and allowed to stir for 16 h, during which time the reaction was monitored by TLC. The mixture was again cooled to 0° C., neutralized by the dropwise addition of 2.0 N HCl, and extracted with three 50 mL portions of ethyl acetate. The ethyl acetate layers were combined, washed with brine, and dried over anhydrous magnesium sulfate. Removal of the solvent in vacuo yielded compound 30d as a cloudy yellow oil (0.375 g, 75.8%) of sufficient purity to be used in the next reaction without further purification. ¹H NMR (400 MHz CDCl₃) δ 1.2 (m, 4H), 1.4 (s, 9H), 1.5 (m, 4H), 2.11 (t, J=7.2 Hz, 2H), 2.28 (t, J=7.2 Hz, 2H), 2.93 (s, 6H), 3.22 (m, 4H), 4.3 (s, 2H), 6.6 (d, J=8.8 Hz, 2H), 7.1 (m, 2H). ¹³C NMR (400 MHz CDCl₃) δ 21.29, 24.57, 25.20, 26.35, 28.72, 29.64, 29.92, 36.19, 39.26, 40.99, 45.76, 50.09, 60.65, 80.01, 113.01, 128.68, 150.09, 171.45, 172.28. IR (cm⁻¹) 3325.4, 2929.7, 2858.4, 1716.8, 1658.4, 1613.0, 1554.6, 1486.8, 1006.0.

7-{4-[N-(tert-Butyloxy)carbonyl]-4-[4-(methyl)benzyl]-amino}butylcarbamoylheptanoic Acid (29e). Compound 29e was synthesized from 28e according to the procedure used to synthesize 29d in 72.7% yield. ¹H NMR (400 MHz CDCl₃) δ 1.18 (m, 6H), 1.39 (bs, 12H), 1.53 (m, 4H), 2.10 (m, 2H), 2.2 (m, 2H), 2.26 (s, 3H), 3.14 (m, 4H), 4.30 (s, 2H), 7.05 (s, 4H) ¹³C NMR (400 MHz CDCl₃) 6, 21.93, 24.93, 25.70, 26.44, 28.62, 28.87, 29.01, 31.07, 36.6, 39.26, 46.05, 49.73, 50.35, 60.58, 64.53, 80.09, 127.34, 127.85, 129.33, 135.45, 136.93, 156.1, 171.38, 173.91. IR (cm⁻¹) 3326.9, 2932.8, 2863.1, 1730.5, 1691.0, 1644.1, 1661.8, 1555.3, 1464.62, 1366.05, 1244.7, 1168.19, 1036.0, 729.15 (cm⁻¹)

7-{4-[N-(tert-Butyloxy)carbonyl]-4-[N,N-(dimethylamino)benzyl]amino}butylcarbamoylheptanoic Acid (29c). Compound 29c was synthesized from 28c according to the procedure used to synthesize 29d in 72.3% yield. ¹HNMR (400 MHz CDCl₃) δ 1.35-1.44 (m, 17H), 1.63 (m, 4H), 2.2 (m, 4H), 2.32 (m, 2H), 2.93 (s, 6H), 3.16 (m, 4H), 4.29 (s, 2H), 6.69 (d, J=8.8 Hz, 2H), 7.11 (m, 2H). ¹³C NMR (400 MHz CDCl₃) δ 14.34, 21.19, 21.93, 24.93, 25.46, 25.76, 26.43, 28.60, 28.88, 29.01, 31.07, 36.6, 39.26, 46.05, 49.73, 50.35, 60.58, 64.53, 80.09, 127.34, 127.85, 129.33, 135.45, 136.93, 156.1, 171.38, 173.91. IR (cm⁻¹) 3348.2, 2929.7, 1718.3, 2658.4, 1616.4, 1555.6, 1478.8, 1398.1, 1110.7.

5-{4-[(tert-Butyloxy)carbonyl]-[4-(methyl)benzyl]-amino}butylcarbamoylpentanoic Acid Methyl Ester (29b). Compound 29b was synthesized from 28b according to the procedure used to synthesize 29d in 70.9% yield. ¹H NMR (400 MHz CDCl₃) δ 1.2 (m, 4H), 1.40 (broad s, 9H), 1.53 (m, 4H), 2.10 (m, 2H), 2.2 (m, 2H), 2.30 (s, 3H), 3.20 (m, 4H), 4.32 (s, 2H), 7.05 (s, 4H). ¹³C NMR (400 MHz CDCl₃) δ 25.70, 26.44, 28.62, 28.87, 29.01, 31.07, 36.6 39.26, 46.05, 49.73, 50.35, 60.58, 64.53, 80.09, 127.34, 127.85, 129.33, 135.45, 136.93, 156.1, 171.38, 173.91. IR (cm⁻¹) 3326.4, 2934.4, 2863.1, 1729.7, 1691.7, 1645.4, 1664.8, 1555.3, 1464.6, 1421.1, 1168.2, 1136.0, 732.4.

5-{4-[(tert-Butyloxy)carbonyl]-[2-(phenyl)benzyl]-amino}butylcarbamoyl}pentanoic Acid Methyl Ester (29a). Compound 29a was synthesized from 28a according to the procedure used to synthesize 29d in 86.1% yield. ¹H NMR (400 MHz CDCl₃) δ 1.30-1.43 (m, 13H), 1.54 (m, 4H), 2.11-2.27 (m, 4H), 2.93-3.05 (m, 4H), 4.36 (d, J=32 Hz, 2H), 7.20-7.38 (m, 9H). ¹³C NMR (400 MHz CDCl₃) δ 21.29, 25.58, 26.54, 28.56, 29.68, 29.92, 30.54, 30.85, 36.26, 39.32, 46.38, 47.24, 48.27, 60.64, 64.60, 80.10, 125.75, 126.77, 127.05, 127.44, 127.85, 128.53, 129.3, 130.18, 135.51, 135.83, 140.97, 141.56, 156.03, 173.62. IR (cm⁻¹) 3304.9, 2921.0, 1730.0, 1671.4, 1651.9, 1551.6, 1158.9.

5-{4-[N-(tert-butyloxy)carbonyl]-4-[N,N-(dimethylamino)benzyl]amino}butylcarbamoyl-pentanohydroxamic Acid (30d). Ethyl chlorformate (0.086 g, 0.0008 mol) and triethylamine (0.08 g, 0.0008 mmol) were added to the solution of 29d (0.300 g, 0.0007 mol) in 5 mL of THF, and the mixture was cooled to 0° C. The reaction was allowed to stir for 20 min, after which time it was filtered and added to 20 mL of freshly prepared 1.76 M hydroxylamine in methanol. The reaction was stirred at room temperature for 30 min, filtered and concentrated in vacuo to yield the crude 30d. Purification on silica gel (ethyl acetate:methanol 4:2) then afforded pure 31d (0.252 g, 77.5%) as a dark yellow oil. ¹H NMR (400 MHz CDCl₃) δ 1.2 (m, 4H), 1.4 (s, 9H), 1.5 (m, 4H), 2.15 (broads, 4H), 2.93 (s, 6H), 3.22 (m, 4H), 3.6 (s, 3H), 4.3 (s, 2H), 6.6 (d, J=8.0 Hz, 2H), 7.1 (m, 2H), 7.8 (broads, NHOH). ¹³C NMR (400 MHz CDCl₃) δ 21.2, 23.11, 25.71, 28.72, 31.51, 39.45, 40.91, 41.08, 45.79, 112.86, 1125.73, 126.25, 128.37, 128.62, 132.28, 150.12, 156.13. IR (cm⁻¹) 3396.8, 2929.7, 1638.9, 1610.2, 1554.6, 1450.8, 1405.4.

7-{4-[N-(tert-butyloxy)carbonyl]-4-[4-(methyl)benzyl]-amino}butylcarbamoylheptanohydroxamic Acid (30e). Compound 30e was synthesized from 29e according to the procedure used to synthesize 30d in 78.0% yield. ¹H NMR (400 MHz CDCl₃) δ 1.24-1.31 (m, 4H), 1.43-1.6 (m, 13H), 2.16 (m, 2H), 2.32 (s, 3H), 3.18 (m, 4H), 4.35 (s, 2H), 7.11 (s, 4H). ¹³C NMR (400 MHz CDCl₃) δ 20.07, 23.29, 25.29, 25.46, 25.57, 26.38, 28.53, 28.61, 32.45, 35.71, 38.24, 46.88, 50.92, 80.01, 117.7, 128.33, 129.64, 129.74, 139.67, 156.22, 174.0.38. IR (cm⁻¹) 3221.6, 2929.7, 2858.4, 1671.4, 1638.9, 1554.6, 1450.8, 1190.1, 1150.8, 1110.9.

7-{4-[N-(tert-Butyloxy)carbonyl]-4-[N,N-(dimethylamino)benzyl]amino}butylcarbamoyl-heptanohydroxamic Acid (30c). Compound 30c was synthesized from 29c according to the procedure used to synthesize 30d in 75.7% yield. ¹H NMR (400 MHz CDCl₃) δ 1.24 (m, 4H), 1.44 (m, 15H), 1.52 (m, 2H), 2.1 (m, 4H), 2.90 (s, 6H), 3.14 (m, 4H), 4.26 (s, 2H), 6.65 (d, J=7.6 Hz, 2H), 7.08 (broad s, ¹H). ¹³C NMR (400 MHz CDCl₃) δ 21.34, 22.43, 23.11, 25.71, 28.72, 31.51, 39.45, 40.91, 41.08, 45.79, 112.86, 1125.73, 126.25, 128.37, 128.62, 132.28, 150.12, 156.13. IR (cm⁻¹) 3385.7, 2932.4, 1632.4, 1622.0.0, 1554.6, 1460.7, 1425.1, 1304.2.

5-{4-[N-(tert-Butyloxy) carbonyl]-4-[4-(methyl)benzyl]-amino}butylcarbamoylpentanohydroxamic Acid (30b). Compound 30b was synthesized from 29b according to the procedure used to synthesize 30d in 80.9% yield. ¹H NMR (400 MHz CDCl₃) δ 1.24-1.31 (m, 4H), 1.43 (m, 9H), 2.18 (m, 4H), 2.32 (s, 3H), 3.18 (m, 4H), 4.32 (s, 2H), 7.11 (s, 4H). ¹³C NMR (400 MHz CDCl₃) δ 25.31, 25.06, 26.38, 28.53, 28.61, 32.45, 35.71, 38.24, 46.88, 50.92, 80.24, 127.37, 127.9, 129.64, 129.74, 137.67, 156.33, 174.21. IR (cm⁻¹) 3267.0, 2929.7, 2858.4, 1651.9, 1548.1, 1463.8, 1418.4.

5-{4-[(tert-butyloxy)carbonyl]-4-[2-(phenyl)benzyl]-amino)butylcarbamoyl}pentanohydroxamic Acid (30a).

Compound 30a was synthesized from 29a according to the procedure used to synthesize 30d in 80.6% yield. ¹H NMR (400 MHz CDCl₃) δ 1.25-1.42 (m, 15H), 1.56 (m, 2H), 1.99-2.26 (m, −4H), 2.94-3.05 (m, 4H), 4.36 (d, J=31.2 Hz, 2H), 7.21-7.40 (m, −9H) ¹³C NMR (400 MHz CDCl₃) δ 25.04, 25.49, 26.47, 28.57, 29.92, 32.24, 35.91, 39.98, 46.5, 48.32, 80.24, 126.8, 125.08, 127.47, 127.88, 128.55, 129.3, 130.2, 135.74, 140.95, 141.58, 156.18, 174.09. IR (cm⁻¹) 3260.5, 2975.1, 2929.7, 1664.9, 1651.9, 1554.6.

5-{4-[4-N,N-(Dimethyl)aminobenzyl]amino}butylcarbamoylpentanohydroxamic Acid (16). Compound 30d (0.140 g, 0.0003 mol) was dissolved in 5 mL of 20% trifluoroacetic acid in dichloromethane, and the reaction was allowed to stir at room temperature for 8 h. The solvent was then removed in vacuo, and the residue was taken up in 25 mL of chloroform and dried over anhydrous magnesium sulfate. Filtration and removal of the solvent afforded crude 16 (0.110 g, 76.7%) as a buff colored solid. An analytical sample of 16 was prepared by recrystallization from aqueous ethanol. ¹H NMR (400 MHz CD₃OD) δ 1.2 (m, 4H), 1.5 (m, 4H), 2.15 (t, J=7.2 Hz, 2H), 2.19 (t, J=7.2 Hz, 2H), 2.93 (s, 6H), 3.22 (m, 4H), 4.03 (s, 2H), 6.7 (d, J=8.0 Hz, 2H), 7.3 (m, 2H). ¹³C NMR (400 MHz CD₃OD) δ23.28, 24.35, 26.31, 131.63, 161.58. IR (cm⁻¹) 2988.1, 1671.4, 1554.6, 1444.3, 1203.8, 1165.9. Anal. for C₂₁H₃₃F₃N₄O₅: C, H, N.

7-{4-[4-(Methyl)benzyl]aminobutyl}carbamoylheptanohydroxamic Acid (17). Compound 17 was synthesized from 30e according to the procedure used to synthesize 16 in 71.6% yield. An analytical sample of 17 was prepared by recrystallization from aqueous ethanol. ¹H NMR (400 MHz CD₃OD) δ 1.23-1.43 (m, 4H), 1.59-1.6 (m, 8H), 2.07-2.17 (m, 4H), 2.35 (s, 3H), 2.86 (t, J=7.2 Hz, 2H), 3.03 (m, 2H), 3.19 (m, 2H), 4.14 (s, 2H), 7.26 (d, J=6.4, 2H), 7.37 (d, J=6.8 Hz, 2H), 8.06 (broad s, NHCO), 9.93 (broad s, NHOH). ¹³C NMR (400 MHz CD₃OD) δ 23.3, 25.4, 25.7, 26.44, 28.54, 28.62, 32.43, 35.68, 38.27, 50.82, 117.70, 128.33, 129.64, 129.8, 139.7, 160.41, 175.21 IR (cm⁻¹) 3226.6, 2929.7, 2853.4, 1671.4, 1640.3, 1551.0, 1444.7, 1209.8, 1163.8. Anal. for C₂₂H₃₄F₃N₃O₅: C, H, N.

7-{4-[4-N,N-(Dimethylamino)benzyl]aminobutyl}-carbamoylheptanohydroxamic Acid (12). Compound 12 was synthesized from 30c according to the procedure used to synthesize 16 in 72.1% yield. An analytical sample of 12 was prepared by recrystallization from aqueous ethanol. ¹H NMR (400 MHz CD₃OD) δ 1.62 (m, 8H), 1.74 (m, 2H), 2.12 (t, J=6.8 Hz, 2H), 2.21 (t, J=6.8 Hz, 2H), 3.06 (m, 2H), 3.15 (s, 6H), 3.21 (m, 2H), 4.20 (s, 2H), 7.36 (broad s, 2H), 7.58 (broad s, 2H). ¹³C NMR (400 MHz CD₃OD) δ 26.86, 26.9, 28.12, 29.01, 29.2, 29.47, 30.13, 30.35, 36.19, 43.09, 46.58, 44.4, 57.0, 112.82, 112.9, 129.32, 129.4, 168.56, 174.23 IR (cm⁻¹) 2982.2, 1671.4, 1556.1, 1445.3, 1203.8, 1168.4. Anal. for C₂₃H₃₇F₃N₄O₅: C, H, N.

5-{4-[4-(Methyl)benzyl]aminobutyl}carbamoylpentanohydroxamic Acid (7). Compound 7 was synthesized from 30b according to the procedure used to synthesize 16 in 72.9% yield. An analytical sample of 7 was prepared by recrystallization from aqueous ethanol. ¹H NMR (400 MHz CD₃OD) δ 1.23-1.43 (m, 4H), 1.6 (m, 4H), 2.0-2.1 (m, 4H), 2.35 (s, 3H), 2.86 (t, J=7.2 Hz, 2H), 3.02 (m, 2H), 3.20 (m, 2H), 4.23 (s, 2H), 7.24 (d, J=6.4, 2H), 7.35 (d, J=6.8 Hz, 2H). ¹³C NMR (400 MHz CD₃OD) δ 26.04, 28.63, 28.54, 34.86, 36.12, 38.99, 51.32, 118.48, 128.33, 129.64, 129.82 139.7, 160.14, 175.21. IR (cm⁻¹) 3221.6, 2929.7, 2858.4, 1781.6, 1671.4, 1638.9, 1554.6, 1450.8, 1190.1, 1150.8, 1110.9. Anal. for C₂₀H₃₀F₃N₃O₅: C, H, N.

5-{4-[2-(Phenyl)benzyl]aminobutyl}carbamoylpentanohydroxamic Acid (5). Compound 5 was synthesized from 30a according to the procedure used to synthesize 16 in 87% yield. An analytical sample of 6 was prepared by recrystallization from aqueous ethanol. ¹H NMR (400 MHz CD₃OD) δ 1.53 (m, −4H), 1.60 (m, 4H), 2.10 (m, 2H), 2.18 (m, 2H), 2.82 (t, J=8 Hz, 2H), 3.11 (t, J=6.4 Hz, 2H), 4.22 (s, 2H), 7.35 (d, J=7.2 Hz, 3H), 7.36-7.50 (m, 5H), 7.66 (d, J=2.8 Hz, ¹H) ¹³C NMR (400 MHz CD₃OD) δ 22.92, 24.9, 25.25, 25.47, 26.2, 32.11, 35.45, 38.10, 47.02, 127.82, 128.31, 1128.70, 128.96, 129.29, 129.47, 130.73, 139.95, 143.27, 155.38, 171.12, 174.79. IR (cm⁻¹) 3723.51, 2923.24, 2851.89, 1671.35, 1561.08, 1431.35, 1176.6, 1112.7. Anal. for C₂₅H₃₂F₃N₃O₅: C, H, N.

1-Phthalimido-4-{N-[2,4,6-(trimethyl)benzenesulfonyl]}-8-{N-[4-(tert-butyl)benzyl]-N-[2,4,6-(trimethyl)benzenesulfonyl]}amino-4-azaoctane (32a). Sodium hydride 60% oil dispersion (equivalent to 0.027 g, 0.0011 mol of NaH was dissolved in 1 mL of dimethylformamide under nitrogen, and the reaction was cooled to 0° C. Compound 31 (0.500 g, 0.0010 mol) was dissolved in 4 mL of dimethylformamide and added dropwise to the reaction mixture, which was allowed to stir for 30 min. A 0.250 g portion (0.0011 mol) of 4-tert-butylbenzyl bromide was dissolved in 2 mL of dimethylformamide and added to the reaction slowly via syringe. The reaction was stirred for 12 h, the solvent was removed in vacuo and the residue was dissolved in water and extracted with three 50 mL portions of ethyl acetate. The combined organic layers were dried over anhydrous magnesium sulfate and filtered, and the solvent was removed to yield crude 32a. Purification of the crude material on silica gel (hexane:ethyl acetate 4:3) then afforded pure 32a as a fluffy white solid (0.619 g, 71.6%). ¹H NMR (400 mHz CDCl₃) δ 1.25 (s, 9H) 1.4 (m, 2H), 1.61 (m, 2H), 1.72 (m, 2H), 2.24 (s, 6H), 2.62 (s, 6H), 2.62 (s, 6H), 3.04-3.06 (t, J=6.4 Hz, 4H), 3.18 (t, J=7.2 Hz, 2H), 4.17 (s, 2H), 6.95 (s, 2H), 7.23 (d, J=6.8 Hz, 2H), 7.73 (m, 2H), 7.83-7.85 (m, 2H). ¹³C NMR (400 MHz CDCl₃) δ 21.19, 23.06, 24.32, 24.49, 24.66, 25.29, 25.39, 25.95, 27.11, 28.70, 31.52, 33.78, 33.87, 36.28, 36.40, 36.47, 43.52, 43.64, 51.76, 51.89, 132.15, 132.30, 132.34, 133.18, 140.15, 140.27, 142.92, 173.24, 174.02, 174.17, 178.84.

1-Phthalimido-4-{N-[2,4,6-(trimethyl)benzenesulfonyl]}-8-{N-[3,3-(diphenyl)propyl]-N-[2,4,6-(trimethyl)-benzenesulfonyl]}amino-4-azaoctane (32b). Compound 32b was synthesized from 31 and 3,3-diphenylpropyl chloride using the procedure described for the synthesis of 32a in 60.3% yield. ¹H NMR (400 MHz CDCl₃) δ 1.40 (m, 4H), 1.69 (q, J=7.2 Hz, 2H), 2.08 (q, J=8 Hz, 2H), 2.3 (s, 3H), 2.44 (s, 3H), 2.51 (d, J=11.2 Hz, 12H), 3.03 (t, J=8 Hz, 2H), 3.09 (t, J=8 Hz, 1H), 3.16 (m, 4H), 3.46 (t, J=6.8 Hz, 2H), 3.73 (t, J=8 Hz, ¹H), 6.76 (s, 2H), 6.90 (s, 2H), 7.03 (d, J=7.6 Hz, 2H), 7.12-7.27 (m, 7H), 7.68 (m, 2H), 7.8 (m, 2H). ¹³C NMR (400 MHz CDCl₃) δ 14.43, 21.18, 21.20, 21.29, 22.96, 22.99, 24.85, 24.90, 26.74, 32.98, 35.43, 44.37, 44.42, 45.40, 45.44, 48.96, 60.62, 123.40, 126.59, 127.76, 128.78, 132.13, 132.18, 132.19, 133.09, 133.4, 134.23, 140.18, 140.35, 142.49, 142.66, 143.99, 168.25. IR (cm⁻¹) 3059.5, 3020.5, 2936.2, 1768.7, 1716.8, 1600.0, 1561.1, 1450.8, 1139.5.

1-Amino-4-{N-[2,4,6-(trimethyl)benzenesulfonyl]}-8-{N-[4-(tert-butyl)benzyl]-N-[2,4,6-(trimethyl)benzenesulfonyl]}amino-4-azaoctane (33a). Compound 33a was prepared from 32a using the procedure described for the synthesis of 26c in 70.8% yield. ¹H NMR (CDCl₃) δ 1.28 (s, 9H) 1.4 (m, 2H), 1.61 (m, 2H), 1.56 (m, 2H), 2.24 (s, 6H), 2.51 (s, 6H), 2.57 (s, 6H), 2.62 (q, J=6.8 Hz, 2H), 3.08 (t, J=7.2 2H), 3.24 (t, J=7.6 Hz, 2H), 3.49 (t, J=7.2 Hz, 2H), 4.2 (s, 2H), 6.81 (s, 2H), 6.95 (m, 6H), 7.23 (d, J=6.8 Hz, 2H). ¹³C NMR (400 MHz CDCl₃) δ 21.11, 21.48, 21.79, 22.5, 22.8, 25.42, 26.87, 28.43, 28.48, 31.74, 32.59, 40.08, 46.77, 46.81, 125.12, 128.2, 136.44, 145.1, 145.64.

1-Amino-4-{N-[2,4,6-(trimethyl)benzenesulfonyl]}-8-(N-[3,3-(diphenyl)propyl]-N-{2,4,6-(trimethyl)benzenesulfonyl]}amino-4-azaoctane (33b). Compound 33b was prepared from 32b using the procedure described for the synthesis of 26c in 78.5% yield. ¹H NMR (400 MHz CDCl₃) δ 1.37 (m, 4H), 1.58 (q, J=7.2 Hz, 2H), 2.04 (m, 2H), 2.28 (s, 3H), 2.31 (s, 3H), 2.46 (s, 6H), 2.57 (s, 6H), 2.97 (t, J=8 Hz, 2H), 3.10 (t, J=4.8 Hz, 2H), 3.17 (m, 4H), 3.48 (t, J=7.2 Hz, ¹H), 3.7 (q, J=5.6 Hz, 2H), 6.9 (d, J=3.2 Hz, 4H), 7.01 (d, J=6.8 Hz, 4H), 7.12-7.23 (m, 5H). ¹³C NMR (400 MHz CDCl₃) δ 14.40, 14.99, 15.84, 21.14, 21.19, 21.26, 22.91, 23.05, 24.66, 25.33, 25.44, 29.90, 31.02, 39.31, 41.28, 42.45, 43.20, 44.63, 45.14, 45.46, 48.90, 52.04, 55.64, 60.06, 64.29, 126.60, 127.67, 128.76, 129.46, 132.18, 133.35, 133.40, 140.22, 140.29, 142.51, 142.60, 143.89, 149.41 IR (cm⁻¹) 3364.3, 3027.0, 2936.2, 2871.4, 1600.0, 1561.1, 1314.6, 1139.5.

15-{N-[2,4,6-(Trimethyl)benzenesulfonyl-N-[(4-tert-butyl)benzyl]}amino-11-[N-2,4,6-(trimethyl)benzenesulfonyl]-6-oxo-7,11-diazapentadecanoic Acid Methyl Ester (34a). Compound 34a was synthesized from 33a and 26a using the method described for the synthesis of 28c in 72.3% yield. ¹H NMR (CDCl₃) δ 1.28 (s, 9H) 1.34 (m, 4H), 1.61 (m, 4H), 1.72 (m, 2H), 2.11 (t, J=7.2 Hz, 2H), 2.24 (m, 8H), 2.51 (s, 6H), 2.62 (s, 8H), 3.08 (m, 2H), 3.24 (m, 2H), 3.6 (t, 3H), 4.13 (s, 2H), 6.90 (broad s, 2H), 7.22 (broad s, 2H), 7.28 (d, 2H). ¹³C NMR (400 MHz CDCl₃) δ 14.35, 14.41, 21.19, 21.27, 22.87, 23.11, 23.12, 24.17, 24.46, 24.68, 25.29, 27.12, 31.50, 31.8, 33.91, 34.72, 36.34, 36.38, 43.29, 44.57, 45.12, 48.93, 51.75, 60.61, 125.74, 128.38, 132.28, 132.30, 132.56, 133.36, 133.39, 140.25, 140.29, 142.80, 151.11, 172.95, 174.11 IR (cm⁻¹) 3387.9, 2952.2, 1738.3, 1655.3, 1603.5, 1541.2, 1458.2.

17-{N-[2,4,6-(Trimethyl)benzenesulfonyl-N-[(4-tert-butyl)benzyl]}amino-13-[N-2,4,6-(trimethyl)benzenesulfonyl]-8-oxo-7,11-diazaheptadecanoic Acid Methyl Ester (34b). Compound 34b was synthesized from 33a and 26b using the method described for the synthesis of 28c in 78.3% yield. ¹H NMR (400 MHz CDCl₃) δ 1.28 (s, 9H), 1.32-1.36 (m, 8H), 1.58-1.7 (m, 8H), 2.09 (t, J=2.8 Hz, 2H), 2.30 (m, 8H), 2.58 (d, J=9.2 Hz, 12H), 3.03 (m, 4H), 3.20 (q, J=6.8 Hz, 6H), 3.6 (s, 3H), 4.12 (s, 2H), 5.979 (t, J=5.6 Hz, ¹H), 6.90 (d, J=8.4 Hz, 2H), 6.96 (s, 4H), 7.27 (d, J=1.2 Hz, 2H). ¹³C NMR (400 MHz CDCl₃) δ 21.19, 23.12, 24.17, 24.46, 24.74, 24.93, 24.97, 25.74, 27.09, 28.89, 28.95, 29.02, 29.10, 31.51, 34.06, 34.2, 34.73, 36.36, 36.76, 43.29, 44.58, 45.13, 48.95, 21.70, 51.72, 125.74, 128.37, 132.28, 132.55, 133.36, 133.39, 140.25, 140.31, 142.81, 151.13, 173.66, 174.45, 178.43. IR (cm⁻¹) 2929.7, 2864.9, 1736.2, 1645.4, 1600.0, 1554.6, 1314.6, 1139.5.

17-{N-[2,4,6-(Trimethyl)benzenesulfonyl-N-[3,3-(diphenyl)propyl]}amino-13-[N-2,4,6-(trimethyl)-benzenesulfonyl]-8-oxo-7,11-diazaheptadecanoic Acid Methyl Ester (34c). Compound 34c was synthesized from 33b and 26b using the method described for the synthesis of 28c in 75.1% yield. ¹H NMR (400 MHz CDCl₃) δ 1.25-1.39 (m, 8H), 1.55-1.69 (m, 6H), 2.05 (q, J=8 Hz, 2H), 2.07 (m, 2H), 2.28 (m, 2H), 2.32 (m, J=8 Hz, 8H), 2.45 (s, 6H), 2.57 (s, 6H), 2.93 (t, J=7.6 Hz, 2H), 3.10 (t, J=7.6 Hz, 2H), 3.15-3.22 (m, 6H), 3.65 (s, 3H), 3.69 (t, J=8 Hz, 1H), 5.84 (t, 1H), 6.91 (d, J=9.6 Hz, 2H), 6.98 (d, J=7.2 Hz, 4H), 7.14-7.20 (m, 5H). ¹³C NMR (400 MHz CDCl₃) δ 14.42, 21.17, 21.20, 21.29, 22.93, 23.10, 24.47, 24.72, 24.98, 25.73, 27.13, 29.04, 29.12, 32.75, 34.20, 36.35, 36.74, 43.32, 44.09, 45.14, 45.29, 48.87, 51.69, 60.29, 126.65, 127.65, 128.79, 132.24, 132.31, 133.36, 140.25, 142.61, 142.79, 143.82, 173.42, 174.42 IR (cm⁻¹) 2936.2, 2851.9, 1736.2, 1671.4, 1638.9, 1593.5, 1146.0.

15-{N-[2,4,6-(Trimethyl)benzenesulfonyl-N-[3,3-(diphenyl)propyl]}amino-11-[N-2,4,6-(trimethyl)-benzenesulfonyl]-6-oxo-7,11-diazapentadecanoic Acid Methyl Ester (34d). Compound 34d was synthesized from 33b and 26a using the method described for the synthesis of 28c in 72.6% yield. ¹H NMR (400 MHz CDCl₃) δ 1.32-1.39 (m, 4H), 1.58-1.60 (m, 4H), 1.68 (q, J=6.8 Hz, 4H), 1.99 (q, J=8 Hz, 2H), 2.10 (t, J=6.8 Hz, 2H), 2.28-2.35 (d, J=9.2 Hz, 8H), 2.45 (s, 6H), 2.57 (s, 6H), 2.93 (t, J=8 Hz, 2H), 3.09 (t, J=9.6 Hz, 2H), 3.15-3.22 (m, 6H), 3.65 (m, 4H), 5.94 (s, 1H), 6.91 (d, J=9.6 Hz, 4H), 6.93 (d, J=6.8 Hz, 4H), 7.12-7.22 (m, 5H). ¹³C NMR (400 MHz CDCl₃) δ 14.41, 21.16, 21.19, 21.83, 22.92, 23.09, 24.46, 24.66, 24.71, 25.28, 27.14, 32.74, 33.91, 36.31, 36.42, 43.34, 44.08, 45.14, 45.28, 48.86, 51.75, 60.62, 126.64, 127.64, 128.79, 132.25, 132.31, 133.36, 140.23, 142.62, 142.81, 143.82, 172.96, 174.12. IR (cm⁻¹) 3370.8, 2929.7, 1736.2, 1671.4, 1651.9, 1600.0, 1541.6.

15-{N-[2,4,6-(Trimethyl)benzenesulfonyl-N-[(4-tert-butyl)benzyl]}amino-11-[N-2,4,6-(trimethyl)benzenesulfonyl]-6-oxo-7,11-diazapentadecanoic Acid (35a). Compound 35a was synthesized from 34a using the method described for the synthesis of 29d in 70.6% yield. ¹H NMR (CDCl₃) δ 1.28 (s, 9H) 1.34 (m, 4H), 1.61 (m, 4H), 1.72 (m, 2H), 2.11 (t, J=8.0 Hz, 2H), 2.24 (m, 8H), 2.51 (s, 6H), 2.62 (s, −8H), 3.08 (m, 2H), 3.24 (m, 2H), 4.13 (s, 2H), 6.90 (broad s, 2H), 7.22 (broad s, 2H), 7.28 (d, J=6.8 Hz, 2H). ¹³C NMR (400 MHz CDCl₃) δ 14.41, 21.27, 23.11, 14.71, 24.46, 24.11, 27.09, 28.97, 29.11, 30.89, 31.50, 34.35, 34.67, 36.54, 36.26, 43.22, 44.05, 45.61, 48.69, 60.46, 64.76, 125.17, 128.53, 132.72, 132.73, 132.15, 133.73, 140.12, 140.43, 142.43, 142.88, 151.81, 171.34, 173.28 IR (cm⁻¹) 3377.3, 2929.7, 1723.2, 1645.4, 1600.0, 1548.1.

17-{N-[2,4,6-(Trimethyl)benzenesulfonyl-N-[(4-tert-butyl)benzyl]}amino-13-[N-2,4,6-(trimethyl)benzenesulfonyl]-8-oxo-7,11-diazaheptadecanoic Acid (35b). Compound 35b was synthesized from 34b using the method described for the synthesis of 29d in 80.0% yield. ¹H NMR (400 MHz CDCl₃) δ 1.27 (s, 9H), 1.32 (m, 8H), 1.56-1.62 (m, 4H), 1.68 (t, J=7.6 Hz, 2H), 2.30 (d, J=5.6 Hz, 8H), 2.57 (d, J=10 Hz, 2H), 3.01 (m, 4H), 3.02-3.23 (m, 4H), 4.12 (s, 2H), 6.05 (s, 1H), 6.90 (d, J=8 Hz, 2H), 6.95 (d, J=3.2 Hz, 4H), 7.25 (d, J=8.4 Hz, 2H). ¹³C NMR (400 MHz CDCl₃) δ 13.92, 14.41, 19.34, 21.19, 21.27, 23.11, 23.12, 24.17, 24.46, 24.81, 25.68, 27.09, 28.91, 28.99, 29.67, 29.91, 30.83, 31.51, 34.37, 34.72, 36.42, 36.69, 43.32, 44.58, 45.13, 48.94, 60.64, 64.60, 125.74, 128.38, 132.28, 132.31, 132.56, 133.37, 140.25, 140.34, 142.81, 151.11, 171.45, 173.81 IR (cm⁻¹) 3370.9, 2929.7, 1723.2, 1645.4, 1600.0, 1549.2.

17-{N-[2,4,6-(Trimethyl)benzenesulfonyl-N-[3,3-(diphenyl)propyl]}amino-13-1N-2,4,6-(trimethyl)-benzenesulfonyl]-8-oxo-7,11-diazaheptadecanoic Acid (35c). Compound 35c was synthesized from 34c using the method described for the synthesis of 29d in 72.8% yield. ¹H NMR (400 MHz CDCl₃) δ 1.25-1.43 (m, 8H), 1.58-1.67 (m, 6H), 1.97-2.08 (m, 4H), 2.27 (d, J=14 Hz, 8H), 2.44 (s, 6H), 2.56 (s, 6H), 2.93 (t, J=8 Hz, 2H), 3.08 (t, J=7.6 Hz, 2H), 3.19 (m, 6H), 3.66 (t, J=7.6 Hz, 1H), 6.00 (t, 1H), 6.92 (d, J=7.6 Hz, 4H), 6.98 (d, J=7.2 Hz, 4H), 7.12-7.20 (m, 5H). ¹³C NMR (400 MHz CDCl₃) δ 14.42, 21.17, 21.20, 21.29, 22.93, 23.01, 24.46, 24.72, 24.82, 25.68, 27.10, 28.91, 28.98, 29.65, 29.92, 30.53, 32.74, 34.45, 36.46, 36.66, 38.74, 43.35, 44.09, 45.13, 45.29, 45.78, 48.86, 60.66, 125.74, 126.65, 127.65, 128.80, 132.32, 133.33, 133.36, 140.24, 142.63, 142.82, 143.83, 171.48, 173.85. IR (cm⁻¹) 3367.2, 2931.4, 172.0, 1645.0, 1593.1, 1541.0.

15-{N-[2,4,6-(Trimethyl)benzenesulfonyl-N-[3,3-(diphenyl)propyl]}amino-11-[N-2,4,6-(trimethyl)-benzenesulfonyl]-6-oxo-7,11-diazapentadecanoic Acid (35d). Compound 35d was synthesized from 34d using the method described for the synthesis of 29d in 80% yield. ¹H NMR (400 MHz CDCl₃) δ 1.41 (m, 4H), 1.43-1.68 (m, 6H), 1.99 (m, 2H), 2.11 (t, J=6.8 Hz, 2H), 2.27 (d, J=17.2 Hz, 6H), 2.44 (s, 6H), 2.58 (s, 6H), 2.93 (t, J=8 Hz, 2H), 3.09 (t, J=6.8 Hz, 2H), 3.20 (m, 6H), 3.66 (t, J=7.6 Hz, 1H), 6.08 (s, 1H), 6.93 (d, J=8 Hz, 4H), 6.98 (d, J=6.8 Hz, 4H), 7.12-7.21 (m, 5H). ¹³C NMR (400 MHz CDCl₃) δ 14.40, 21.16, 21.19, 21.27, 22.92, 23.08, 24.45, 24.71, 25.14, 27.098, 29.91, 32.72, 33.78, 36.22, 36.54, 43.42, 44.06, 45.18, 45.28, 48.84, 60.64, 126.64, 127.63, 128.78, 132.26, 132.31, 133.30, 140.24, 142.65, 142.81, 143.81, 171.45, 173.37, 177.76. IR (cm⁻¹) 3364.3, 2936.2, 1723.2, 1710.3, 1632.4, 1600.0, 1314.6, 1152.4.

15-{N-{2,4,6-(Trimethyl)benzenesulfonyl-N-[(4-tert-butyl)benzyl]}amino-11-[N-2,4,6-(trimethyl)benzenesulfonyl]-6-oxo-7,11-diazapentadecanohydroxamic Acid (36a). Compound 36a was synthesized from 35a according to the method described for the synthesis of 30d in 76.6% yield. ¹H NMR (CDCl₃) δ 1.27 (broad s, 13H), 1.61 (m, 4H), 2.16 (broad s, 2H), 2.3 (m, 8H), 2.54 (s, 6H), 2.62 (s, 8H), 2.99 (m, 4H), 3.24 (m, 4H), 4.11 (s, 2H), 6.87 (d, J=6.8 Hz, 2H), 6.95 (s, 4H) 7.28 (d, J=7.2 Hz, 2H). ¹³C NMR (400 MHz CDCl₃) δ 21.18, 23.10, 24.14, 24.40, 24.88, 27.28, 31.50, 34.71, 35.88, 36.83, 43.52, 44.58, 45.15, 48.89, 50.89, 125.72, 128.37, 132.30, 132.52, 133.23, 133.30, 140.23, 140.28, 142.85, 151.09, 174.16. IR (cm⁻¹) 3340.5, 2936.2, 2858.4, 1716.8, 1644.5, 1600.0, 1544.2, 1457.3.

17-{N-[2,4,6-(Trimethyl)benzenesulfonyl-N-[(4-tert-butyl)benzyl]}amino-13-[N-2,4,6-(trimethyl)benzenesulfonyl]-8-oxo-7,11-diazaheptadecanohydroxamic Acid (36b). Compound 36b was synthesized from 35b using the method described for the synthesis of 30d in 65.9% yield. ¹H NMR (400 MHz CDCl₃) δ 1.27 (s, 9H), 1.31 (m, 8H), 1.61-1.69 (m, 4H), 2.1 (m, 3H), 2.29-2.31 (m, s, 7H), 2.58 (d, J=7.6 Hz, 12H), 3.02 (m, 4H), 3.23 (m, 4H), 4.10 (s, 2H), 6.88 (d, J=7.6 Hz, 2H), 6.96 (s, 4H), 7.24 (s, 2H). ¹³C NMR (400 MHz CDCl₃) δ 20.97, 21.21, 23.12, 24.18, 24.46, 31.51, 34.74, 44.59, 45.21, 48.91, 125.71, 128.31, 132.34, 132.4, 132.5, 133.3, 140.26, 140.21, 142.88, 151.15, 174.12. IR (cm⁻¹) 3247.6, 2936.2, 2858.4, 1716.8, 1646.4, 1606.5, 1541.6, 1457.3.

17-{N-[2,4,6-(Trimethyl)benzenesulfonyl-N-[3,3-(diphenyl)propyl]}amino-13-[N-2,4,6-(trimethyl)-benzenesulfonyl]-8-oxo-7,11-diazaheptadecanohydroxamic Acid (36c). Compound 36c was synthesized from 35c using the method described for the synthesis of 30d in 75.8% yield. ¹H NMR (400 MHz CDCl₃) δ 1.24-1.39 (m, 8H), 1.54-1.68 (m, 6H), 2.10 (m, 4H), 2.24 (s, 4H), 2.30 (s, 4H), 2.43 (s, 6H), 2.54 (s, 6H), 2.9 (m, 2H), 3.03 (m, 2H), 3.21 (m, 6H), 3.66 (t, 1H), 6.90 (s, 4H), 6.97 (d, J=6.4 Hz, 4H), 7.13-7.19 (m, 6H). ¹³C NMR (400 MHz CDCl₃) δ 14.42, 21.21, 22.92, 23.10, 24.37, 24.70, 32.74, 43.42, 44.11, 45.31, 48.83, 60.65, 126.62, 127.66, 128.78, 132.26, 132.34, 133.33, 140.19, 140.24, 142.62, 142.81, 143.86, 171.45, 174.80. IR (cm⁻¹) 3254.1, 2929.7, 2858.4, 1651.9, 1632.4, 1606.5, 1554.6.

15-{N-[2,4,6-(Trimethyl)benzene sulfonyl-N-[3,3-(diphenyl)propyl]}amino-11-[N-2,4,6-(trimethyl)-benzenesulfonyl]-6-oxo-7,11-diazapentadecanohydroxamic Acid (36d). Compound 36d was synthesized from 35d using the method described for the synthesis of 30d in 70.2% yield. ¹H NMR (400 MHz CDCl₃) δ 1.39 (m, 4H), 1.59-1.68 (m, 6H), 2.01 (m, 4H), 2.20 (s, 3H), 2.25 (s, 3H), 2.43 (s, 6H), 2.54 (s, 6H), 2.91 (m, 2H), 3.05 (m, 2H), 3.17 (m, 6H), 3.64 (t, J=7.6 Hz, 1H), 6.90 (s, 4H), 6.98 (d, J=7.2 Hz, 2H), 7.12-7.26 (m, 5H). ¹³C NMR (400 MHz CDCl₃) δ 14.42, 21.20, 21.29, 22.97, 23.08, 24.68, 29.93, 32.71, 44.08, 45.30, 48.83, 52.55, 60.64, 126.64, 127.65, 128.79, 132.27, 133.31, 140.23, 142.67, 143.83, 162.23, 171.44. IR (cm⁻¹) 3340.5, 1638.9, 1606.6, 1535.1, 1444.3, 1308.1.

15-N-[(4-tert-Butyl)benzyl]amino-6-oxo-7,11-diazapentadecanohydroxamic Acid dihydrobromide (11). A 4.71 g portion of phenol (0.050 mol) was dissolved in 50 mL of 30% HBr in acetic acid in a stoppered flask, and to this mixture was added a solution of 36a (0.300 g, 0.0004 mol) in 20 mL of ethyl acetate in three portions over a period of 3 h After the addition was complete, the reaction mixture was stirred for an additional 15 h at room temperature, then cooled to 0° C., and diluted with 100 mL of water. The aqueous phase was washed with two 100 mL portions of ethyl acetate before being lyophylized to give the crude product as yellow solid. This crude product was washed with methanol and filtered to yield the tetrahydrobromide salt of 11 (0.186 g, 77.8%) as an off white solid. An analytical sample of 11 was prepared by recrystallization from aqueous ethanol. ¹H NMR (D₂O) δ 1.20 (s, 9H), 1.42 (m, 4H) 1.61 (m, 4H), 2.01 (m, 2H), 2.13 (m, 2H), 2.99 (m, 9H), 3.12 (m, 2H), 4.06 (s, 2H), 7.26 (d, J=6.4 Hz, 2H), 7.24 (d, J=6.4 Hz 2H). ¹³C NMR (400 MHz D₂O) δ 22.85, 22.92, 22.98, 23.81, 24.06, 24.52, 24.78, 25.77, 30.55, 32.13, 34.26, 35.42, 36.06, 38.93, 45.21, 46.20, 47.00, 47.08, 50.73, 126.44, 127.83, 129.94, 130.13, 153.57, 173.06, 177.18. IR (cm⁻¹) 3419.0, 2952.2, 1696.8, 1686.5, 1634.6, 1603.5, 1551.6. Anal. for C₂₄H₄₄Br₂N₄O₃: C, H, N.

17-N-[(4-tert-Butyl)benzyl]amino-8-oxo-7,11-diazaheptadecanohydroxamic Acid dihydrobromide (15). Compound 15 was synthesized from 36b using the method de-scribed for the synthesis of 11 in 78.4% yield. An analytical sample of 15 was prepared by recrystallization from aqueous ethanol. ¹H NMR (400 MHz D₂O) 1.15-1.28 (m, 13H), 1.62 (m, 4H), 1.74 (m, 2H), 1.99 (m, 2H), 2.10 (m, 2H), 2.91 (m, 8H), 3.14 (m, 2H), 4.07 (d, J=10.08 Hz, 2H), 7.29 (t, J=8 Hz, 2H), 7.42 (t, J=9.6 Hz, 2H). ¹³C NMR (400 MHz D₂O) δ 22.84, 24.48, 25.23, 25.77, 27.76, 27.91, 30.54, 32.38, 35.70, 36.00, 45.19, 46.16, 47.00, 50.71, 126.44, 127.83, 129.94, 130.13, 153.44, 173.41, 177.85. IR (cm⁻¹) 3240.5, 2936.2, 1682.2, 1645.4, 1603.2, 1541.6, 1457.3. Anal. for C₂₆H₄₈Br₂N₄O₃: C, H, N.

17-N-[4-(3,3-Diphenyl)propyl]amino-8-oxo-7,11-diazaheptadecanohydroxamic Acid dihydrobromide (19). Compound 19 was synthesized from 36c using the method de-scribed for the synthesis of 11 in 75.7% yield. An analytical sample of 19 was prepared by recrystallization from aqueous ethanol. ¹H NMR (400 MHz D₂O) δ 1.07 (m, 4H), 1.39 (m, 4H), 1.53 (m, 4H), 1.68 (m, 2H), 1.96 (m, 2H), 2.06 (m, 2H), 2.30 (m, 2H), 2.83 (m, 8H), 3.09 (m, 2H), 3.94 (m, 1H), 7.11 (m, −1H), 7.22 (m, 8H). ¹³C NMR (400 MHz D₂O) δ 20.61, 22.76, 22.86, 24.84, 22.86, 24.84, 25.20, 25.75, 27.73, 27.89, 30.85, 32.33, 35.65, 35.93, 45.12, 45.43, 46.75, 46.94, 48.08, 61.82, 63.38, 127.15, 127.58, 129.18, 143.64, 173.58, 177.85 IR (cm⁻¹) 3390.3, 2923.2, 2845.4, 1658.4, 1632.4, 1554.6, 1444.3. Anal. for C₃₀H₄₈Br₂N₄O₃: C, H, N.

15-N-[4-(3,3-diphenyl)propyl]amino-6-oxo-7,11-diazapentadecanohydroxamic Acid dihydrobromide (20). Compound 20 was synthesized from 36d using the method de-scribed for the synthesis of 11 in 70.1% yield. An analytical sample of 20 was prepared by recrystallization from aqueous ethanol. ¹H NMR (400 MHz D₂O) δ 1.07 (m, 4H), 1.39 (m, 4H), 1.53 (m, 4H), 1.68 (m, 2H), 1.97 (m, 2H), 2.06 (m, 2H), 2.30 (m, 2H), 2.83 (m, 8H), 3.01 (m, 2H), 3.94 (m, 2H), 7.11 (m, 1H), 7.22 (m, 8H). ¹³C NMR (400 MHz D₂O) δ 22.8, 22.89, 24.51, 24.78, 25.79, 30.89, 35.42, 36.02, 45.12, 45.18, 46.49, 46.72, 46.81, 46.97, 48.13, 127.18, 127.18, 127.62, 129.22, 143.68, 177.23. IR (cm⁻¹) 3383.8, 2903.8, 1658.4, 1632.4, 1561.1, 1444.3. Anal. for C₂₈H₄₄Br₂N₄O₃: C, H, N.

1-(Phthalimido)-4,8,12-tris{N-[2,4,6-(trimethyl)benzenesulfonyl]}-15-N-[2,4,6-trimethylbenzenesulfonyl)-amino]-4,8,12-triazapentadecane (38). Compound 38 was synthesized from 37 using the procedure described for the synthesis of 32a in 65.7% yield. ¹H NMR (CDCl₃) δ 1.22 (m, 4H), 1.33-1.44 (m, 2H), 1.61-1.72 (m, 2H), 2.22 (s, 12H), 2.53 (s, 12H), 2.58 (s, 12H), 2.8 (q, 2H), 2.95 (m, 8H), 3.2 (t, J=7.2 Hz, 2H), 6.99 (d, J=6.8 Hz, 8H), 7.73 (m, 2H), 7.83-7.85 (m, 2H).

1-(Phthalimido)-4,8,12-tris{N-[2,4,6-(trimethyl)benzenesulfonyl]}-15-{N-[2,4,6-(trimethyl)benzenesulfonyl]-N-[2-(phenyl)benzyl]}amino-4,8,12-triazapentadecane (39a)

1-(Phthalimido)-4,8,12-tris{N-[2,4,6-(trimethyl)benzenesulfonyl]}-15-{N-[2,4,6-(trimethyl)benzenesulfonyl]-N-[(cyclopropyl)methyl]}amino-4,8,12-triazapentadecane (39b)

1-(Phthalimido)-4,8,12-tris(N-[2,4,6-(trimethyl)benzenesulfonyl])-15-{N-[2,4,6-(trimethyl)benzenesulfonyl]-N-[(cycloheptyl)methyl]}amino-4,8,12-triazapentadecane (39c)

Compounds 39a-c were synthesized from 38 and the appropriate alkyl halide using the method described for the synthesis of 32a.

Compound 39a (65% yield). ¹H NMR (400 MHz CDCl₃) δ 1.08 (m, 2H), 1.48 (m, 2H), 1.62 (m, 4H), 2.21 (m, 12H), 2.43 (s, 6H), 2.46 (s, 6H), 2.52 (m, 14H), 2.64 (t, J=6.8 Hz, 2H), 2.75 (t, J=7.2 Hz, 2H), 2.91-3.07 (m, 8H), 3.42 (t, J=6.8 Hz, 2H), 4.27 (s, 2H), 6.77 (s, 2H), 6.85 (s, 2H), 6.91 (s, 2H), 6.94 (s, 2H), 7.16 (m, 3H), 7.26 (m, 3H), 7.38 (m, 3H), 7.72 (m, 2H), 7.80 (m, 2H).

Compound 39b (73% yield). ¹H NMR (CDCl₃) δ 0.02 (m, 2H), 0.45 (dd, J=8 Hz, 5.2 Hz, 2H), 0.75 (m, 1H), 1.66 (m, 8H), 2.18 (s, 2H), 2.25 (s, 2H), 2.30 (s, 8H), 2.48 (s, 6H), 2.56 (s, 18H), 2.91 (d, J=6.8 Hz, 2H), 3.01 (m, 12H), 3.15 (t, J=7.2 Hz, 2H), 3.44 (t, J=6.8 Hz, 2H), 6.79 (s, 2H), 6.94 (s, 6H), 7.76 (m, 2H), 7.82 (m, 2H).

Compound 39c (72.2% yield). ¹H NMR (400 MHz CDCl₃) δ 0.77 (m, 2H), 1.22 (m, 8H), 1.33-1.44 (m, 6H), 1.61-1.72 (m, 6H), 2.22 (s, 12H), 2.53 (s, 12H), 2.58 (s, 12H), 2.7 (d, J=6.8 Hz, 2H) 2.8 (q, J=6.6 Hz, 2H), 2.95 (m, 12H), 3.2 (t, J=7.2 Hz, 2H), 6.99 (d, J=7.6 Hz, 8H), 7.73 (m, 2H), 7.83-7.85 (m, 2H).

11,15,19-Tris{N-[2,4,6-(trimethyl)benzenesulfonyl]}-22-(N-[2,4,6-trimethylbenzenesulfonyl)]-N-[2-(phenyl)-benzyl]amino)-6-oxo-7,11,15,19-tetraazadocosanoic Acid Methyl Ester (40a)

11,15,19-iris {N-[2,4,6-(trimethyl)benzenesulfonyll}-22-{N-[2,4,6-trimethylbenzenesulfonyl)]-N-[(cyclopropyl)-methyl]amino}-6-oxo-7,11,15,19-tetraazadocosanoic Acid Methyl Ester (40b)

11,15,19-Tris {N-[2,4,6-(trimethyl)benzenesulfonyl]}-22-{N-[2,4,6-trimethylbenzenesulfonyl)]-N-[2-(cycloheptyl)-methyl]amino}-6-oxo-7,11,15,19-tetraazadocosanoic Acid Methyl Ester (40c)

Compounds 40a-c were synthesized from 39a-c and 27a in two steps using the methods described for the synthesis of 26a and 28a.

Compound 40a (70% yield). ¹H NMR (CDCl₃) δ 1.08 (m, 2H), 1.48 (m, 2H), 1.43-1.79 (m, 8H), 2.05 (m, 2H), 2.21-2.39 (m, 14H), 2.53 (m, 28H), 2.77 (t, J=7.2 Hz, 2H), 2.98 (m, 8H), 3.12 (m, 4H), 3.61 (s, 3H), 4.19 (s, 2H), 6.91 (s, 2H), 6.94 (m, 6H), 7.13 (m, 4H), 7.24 (m, 3H), 7.35 (m, 2H). ¹³C NMR (400 MHz CDCl₃) δ 21.18, 22.98, 23.02, 23.05, 23.08, 24.26, 24.68, 25.11, 25.28, 25.41, 25.78, 27.13, 33.91, 34.02, 36.25, 42.75, 43.25, 43.28, 46.18, 51.75, 109.99, 127.69, 127.78, 128.98, 128.65, 129.19, 129.46, 130.29, 132.18, 132.29, 132.34, 132.85, 133.06, 140.06, 140.26, 140.44, 142.46, 142.80, 142.89, 142.95, 172.45, 174.32. IR (cm⁻¹) 3336.0, 2931.4, 1740.5, 1655.3, 1582.7.

Compound 40b (74% yield). ¹HNMR (CDCl₃) δ 0.01 (m, 2H), 0.46 (m, 2H), 0.75 (m, 1H), 1.58 (m, 12H), 2.01 (m, 2H), 2.32 (s, 12H), 2.43 (m, 2H), 2.68 (s, 24H), 2.92 (d, J=6.8 Hz, 2H), 3.06 (m, 10H), 3.18 (m, 4H), 3.78 (s, 3H), 6.98 (m, 8H). IR (cm⁻¹) 3383.7, 1742.7, 1664.8, 1606.4, 1561.0.

Compound 40c (70.2% yield). ¹H NMR (CDCl₃) δ 0.77 (m, 2H), 1.22 (m, 2H), 1.33-1.44 (m, 8H), 1.61-1.78 (m, 12H), 2.1 (t, 2H), 2.22 (m, 14H), 2.53 (s, 12H), 2.58 (s, 14H), 2.75 (d, J=6.8 Hz, 2H), 2.9-3.01 (m, 12H), 3.14-3.18 (m, 4H), 3.67 (s, −3H), 6.88-6.94 (m, 8H). ¹³C NMR (400 MHz CDCl₃) δ 21.19, 23.06, 24.32, 24.49, 24.66, 25.29, 25.39, 25.95, 27.11, 28.70, 31.52, 33.78, 33.87, 36.28, 36.40, 36.47, 43.45, 43.52, 43.64, 51.76, 51.89, 132.15, 132.30, 132.34, 133.18, 140.15, 140.27, 142.95, 173.24, 174.02, 174.17, 178.84.

11,15,19-Tris{N-[2,4,6-(trimethyl)benzenesulfonyl]}-22-{N-[2,4,6-trimethylbenzenesulfonyl)]-N-[2-(phenyl)benzyl]amino}-6-oxo-7,11,15,19-tetraazadocosanohydroxamic Acid (41a)

11,15,19-Tris {N-[2,4,6-(trimethyl)benzenesulfonyl]}-22-{N-[2,4,6-trimethylbenzenesulfonyl)]-N-[(cyclopropyl)-methyl]amino}-6-oxo-7,11,15,19-tetraazadocosanohydroxamic Acid (41b)

11,15,19-Tris {N-[2,4,6-(trimethyl)benzenesulfonyl]}-22-{N-[2,4,6-trimethylbenzenesulfonyl)]N-[2-(cycloheptyl)-methyl]amino}-6-oxo-7,11,15,19-tetraazadocosanohydroxamic Acid (41c)

Compounds 41a-c were synthesized from 40a-c in two steps using the methods described for the synthesis of 29a and 30a.

Compound 41a (62.6% yield). ¹H NMR (CDCl₃) δ 1.41-1.79 (m, 12H), 2.13 (m, 2H), 2.21-2.38 (m, 14H), 2.40-2.59 (m, 28H), 2.78 (t, J=7.2 Hz, 2H), 2.98 (m, 6H), 3.18 (m, 2H), 4.20- (8, 2H), 6.88 (s, 2H), 6.96 (m, 6H), 7.15-7.43 (m, 9H) ¹³C NMR (400 MHz CDCl₃) δ 14.42, 21.13, 21.18, 21.21, 21.28, 23.02, 23.09, 24.15, 24.40, 27.61, 31.61, 39.57, 42.39, 43.25, 43.82, 45.24, 46.15, 51.71, 60.61, 110.12, 127.66, 127.77, 127.99, 128.64, 129.19, 129.48, 130.27, 132.14, 132.33, 133.08, 133.31, 134.19, 139.11, 140.03, 140.17, 140.29, 140.45, 142.12, 142.46, 142.62, 142.83, 142.93, 168.21, 174.13 IR (cm⁻¹) 3407.6, 2910.1, 2765.4, 1664.7, 1644.9, 1603.6, 1447.84.

Compound 41b (60.9% yield). ¹H NMR (CDCl₃) δ 0.01 (m, 2H), 0.5 (m, 2H), 0.72 (m, 1H), 1.73 (m, 12H), 2.29 (m, 2H), 2.30 (m, 2H), 2.41 (s, 12H), 2.65 (s, 24H), 2.88 (d, J=6.8 Hz, 2H), 3.04 (t, J=7.6 Hz, 2H), 3.10 (m, 8H), 3.21 (t, J=7.2 Hz, 2H), 3.28 (m, 4H), 7.06 (s, 8H). ¹³C NMR (400 MHz CDCl₃) δ 5.05, 11.08, 17.20, 18.02, 18.96, 19.05, 19.09, 20.71, 21.30, 21.55, 25.94, 31.74, 32.63, 46.13, 128.21, 128.31, 128.35, 128.96, 129.07, 129.21, 136.29, 138.75, 139.0, 150.92, 169.62, 172.23. IR (cm⁻¹) 3312.0, 2904.4, 1658.2, 1645.4, 1605.8, 1540.9.

Compound 41c (75.9% yield). ¹H NMR (CDCl₃) δ 0.77 (m, 4H), 1.33-1.44 (m, 8H), 1.61-1.78 (m, 12H), 2.15-2.27 (m, 4H), 2.22 (s, 12H), 2.53 (s, 12H), 2.58 (s, 12H), 2.66 (d, J=7.2 Hz, 2H), 2.9-3.01 (m, 12H), 3.14-3.18 (m, 4H), 6.92-6.98 (m, 8H). ¹³C NMR (400 MHz CDCl₃) δ 14.42, 21.19, 21.29, 23.02, 23.06, 24.72, 24.88, 25.36, 25.94, 28.71, 36.44, 43.38, 43.56, 60.64, 64.60, 132.15, 132.34, 132.98, 133.10, 133.33, 133.97, 140.14, 140.26, 142.70, 142.97, 171.44, 173.82.

22-{N-[2-(Phenyl)benzyl]amino}-6-oxo-7,11,15,19-tetraazadocosanohydroxamic Acid (6). Compound 7 was synthesized from 41a using the method described for the synthesis of 11 in 68% yield. ¹H NMR (D₂O) δ 1.48 (m, 4H), 1.79 (m, 4H), 1.98 (m, 4H), 2.15 (m, 2H), 2.23 (m, 2H), 2.75 (t, J=8.0 Hz, 2H), 2.8-3.05 (m, 12H), 3.12 (t, J=6.4 Hz, 2H), 4.16 (s, 2H), 7.24-7.44 (m, 9H). ¹³C NMR (400 MHz D₂O) δ 22.42, 22.76, 22.83, 23.75, 23.81, 24.83, 25.77, 33.42, 33.51, 35.47, 36.06, 43.92, 44.62, 44.67, 45.44, 48.23, 115.47, 120.78, 128.05, 128.21, 128.67, 129.10, 129.56, 129.90, 130.08, 130.12, 130.97, 139.53, 142.81, 177.23, 178.65. IR (cm⁻¹) 3419.0, 3045.5, 2910.6, 1688.5, 1651.3, 1603.4. Anal. for C₃¹H₅₄Br₄N₆O₃: C, H, N.

22-{N-[(Cyclopropyl)methyl]amino}-6-oxo-7,11,15,19-tetraazadocosanohydroxamic Acid (8). Compound 9 was synthesized from 41b using the method described for the synthesis of 12 in 65% yield. ¹H NMR (D₂O) δ 0.22 (m, 2H), 0.54 (m, 2H), 0.91 (m, 1H), 1.45 (m, 5H), 1.75 (q, J=6.8 Hz, 3H), 1.98 (m, 4H), 2.13 (t, J=6.8 Hz, 2H) 2.25 (t, J=7.2 Hz, 2H), 2.83 (d, J=7.6 Hz, 2H), 2.93 (t, J=7.6 Hz, 2H), 3.05 (m, 12H), 3.14 (t, J=7.2 Hz, 2H). IR (cm⁻¹) 3312.1, 2931.4, 1655.3, 1645.4, 1593.0. Anal. for C₂₂H₅₀Br₄N₆O₃: C, H, N.

22-(N-[2-(Cycloheptyl)methyl]amino)-6-oxo-7,11,15,19-tetraazadocosanohydroxamic Acid (9). Compound 9 was synthesized from 41c using the method described for the synthesis of 11 in 70.4% yield ¹H NMR (D₂O) δ 1.08-1.16 (m, 4H), 1.28-1.58 (4, 14H), 1.73-1.78 (m, 2H), 1.93-2.07 (m, 6H), 2.78 (d, J=8.8 Hz, 2H), 2.93 (t, J=7.2 Hz, 2H) 2.96-3.01 (m, 12H), 3.12-3.16 (t, J=6.8 Hz, 2H). ¹³C NMR (400 MHz D₂O) δ 22.66, 22.79, 24.52, 24.76, 25.42, 25.81, 27.95, 31.25, 32.12, 35.42, 36.01, 36.30, 173.11, 177.26 IR (cm⁻¹) 3408.7, 2910.7, 2848.4, 1665.7, 1644.0, 1447.8. Anal. for C₂₆H₅Br₄N₆O₃: C, H, N.

7-{4-[(Phthalimido)amino]butyl}-{N-[4-N,N-(dimethyl)aminobenzyl]carbamoyl}heptanoic Acid Methyl Ester (42). Compound 42 was synthesized from 24c and 27b using the procedure described for the synthesis of 28c in 72.6% yield. ¹H NMR (400 MHz CDCl₃) δ 1.24-1.35 (m, 4H), 1.61-1.64 (m, 8H), 2.26-2.36 (m, 4H), 2.92 (d, J=8.8 Hz, 6H), 3.20 (t, J=7.2 Hz, 1H), 3.36 (t, J=6.8 Hz, 1H), 3.65-3.71 (m, 5H), 4.42 (s, 1H), 4.48 (s, 1H), 6.65 (d, J=8.8 Hz, 1H), 6.69 (d, J=8.4 Hz, 1H), 7.01 (d, J=7.6 Hz, 1H), 7.11 (d, J=8.4 Hz, 1H), 7.69-7.75 (m, 2H), 7.80-7.86 (m, 2H). ¹³C NMR (400 MHz CDCl₃) δ 24.82, 24.99, 25.56, 26.21, 28.92, 28.97, 29.20, 29.29, 33.24, 33.40, 34.19, 37.57, 37.86, 40.81, 40.86, 45.39, 46.23, 47.62, 50.79, 51.65, 112.84, 113.00, 123.37, 123.50, 124.50, 125.84, 127.47, 129.46, 132.19, 134.08, 134.26, 150.11, 150.21, 168.57, 173.08, 174.45 IR (cm⁻¹) 3461.6, 2936.2, 2858.4, 1742.7, 1768.7, 1710.3, 1638.9, 1613.0, 1515.7.

7-{4-[N-(tert-Butyloxycarbonyl)amino]butyl}-{N-[4N,N-(dimethyl)aminobenzyl]carbamoyl}heptanoic Acid (43). Compound 43 was synthesized from 42 in three steps using the procedures described for the synthesis of 26b (phthalimide cleavage) and 25c (N-Boc protection) and 30c in 70.2% overall yield. ¹H NMR (400 MHz CDCl₃) δ 1.25 (m, 2H), 1.30 (m, 2H), 1.39 (s, 12H), 1.47 (m, 2H), 1.58 (m, 4H), 2.15 (m, 2H), 2.29 (m, 2H), 2.86 (d, J=7.2, 6H), 3.03 (m, 2H), 3.11 (t, J=7.6, 1H), 3.26 (t, J=7.2 Hz, 1H), 4.35 (s, 1H), 4.42 (s, 1H), 4.85 (s, 1H), 6.60 (d, J=8.8 Hz, 1H), 6.64 (d, J=8.8 Hz, 1H), 6.96 (d, J=8.4 Hz, 1H), 7.04 (d, J=8.8 Hz, 1H). ¹³C NMR (400 MHz CDCl₃) δ 14.32, 21.24, 24.24, 25.28, 25.36, 25.86, 27.54, 28.36, 28.62, 28.95, 33.30, 33.94, 40.25, 40.39, 40.84, 40.89, 45.56, 46.47, 47.63, 50.77, 60.60, 81.44, 112.90, 113.09, 124.51, 127.49, 129.38, 150.08, 150.25, 156.30, 171.42, 173.24, 173.32, 173.70. IR (cm⁻¹) 3280.0, 2975.1, 2929.7, 2864.9, 1690.8, 1619.5, 1522.2.

7-{4-[N-(tert-Butyloxycarbonyl)amino]butyl}-{N-[4 N,N-(dimethyl)aminobenzyl]carbamoyl}heptano-hydroxamic Acid (44). Compound 44 was synthesized from 43 using the procedure described for the synthesis of 30c in 74.4% yield. ¹H NMR (400 MHz CDCl₃) δ 1.31 (m, 4H), 1.37 (m, 4H), 1.64 (m, 4h), 2.29-2.36 (m, 4H), 2.94 (d, J) 8.4 Hz, 6H), 3.09 (m, 2H), 3.27 (t, J=7.2 Hz, 1H), 3.32 (t, J=8.6 Hz, 1H), 4.41 (s, 1H), 4.48 (s, 1H), 6.66 (d, J=8.4 Hz, 1H), 6.72 (d, J=8.8 Hz, 1H), 7.02 (d, J=8.8 Hz, 1H), 7.1 (d, J=8.8 Hz, 1H), 8.22 (d, J=8.8 Hz, NH). ¹³C NMR (400 MHz CDCl₃) δ 25.00, 25.26, 25.35, 25.88, 27.60, 28.37, 28.64, 28.92, 29.90, 33.00, 33.27, 34.01, 40.28, 40.44, 40.87, 40.91, 45.58, 46.47, 47.65, 50.79, 81.63, 112.89, 113.10, 124.55, 127.50, 129.44, 150.12, 150.27, 155.91, 156.27, 173.19, 173.67, 174.97. IR (cm⁻¹) 3267.0, 2975.1, 2936.2, 2864.9, 1684.3, 1677.8, 1619.5, 1509.2, 1450.8.

7-{[4-(Amino)butyl]-N-[4-(dimethylaminobenzyl)-carbamoyl]}heptanohydroxamic Acid (18). synthesized from 31 according to the procedure mentioned for 12 in 90.9% yield. ¹H NMR (400 MHz CD³OD) δ 1.31-1.40 (m, 4H), 1.64 (m, 8H), 2.26-2.36 (m, 2H), 2.38 (t, J=4.8 Hz, 1H), 2.49 (t, J=4.8 Hz, 1H), 2.94 (m, 2H), 3.29 (s, 6H), 3.39 (m, 2H), 4.65 (s, 1H), 4.73 (s, 1H), 7.45 (t, J=7.2 Hz, 2H), 7.62 (d, J=6 Hz, 1H), 7.67 (d, J=5.6 Hz, 1H). ¹³C NMR (400 MHz CD₃-OD) δ 24.14, 24.59, 24.72, 24.77, 24.97, 25.13, 25.42, 28.62, 28.68, 28.78, 32.52, 32.86, 39.11, 50.33, 114.58, 117.45, 120.59, 120.94, 128.400, 129.46, 139.73, 140.57, 141.98, 160.22, 173.05, 174.63, 175.03. IR (cm⁻¹) 2936.2, 1671.4, 1626.0, 1515.7, 1463.8, 1424.8. Anal. for C₂₃H³⁷F³N⁴O⁵: C, H, N.

1-N-[2,4,6-(Trimethyl)benzenesulfonyl]amino-4-N-[2,4,6-(trimethyl)benzenesulfonyl]-8-N-{4-[N,N-(dimethyl)-benzyl]amino}-4-azaoctane (45). Compound 45 was synthesized in two steps from 31 using the procedure described for the syntheses of 26c and 24c in 77.3% overall yield. ¹H NMR (400 MHz CDCl₃) δ 1.35 (q, J=7.6 Hz, 2H), 1.47 (q, J=6.8 Hz, 2H), 1.57 (q, J=6.8 Hz, 2H), 2.28 (d, J=2.8 Hz, 6H), 2.51-2.59 (m, 14H), 2.78 (t, J=6.8 Hz, 2H), 3.13 (t, J=7.6 Hz, 2H), 3.17 (t, J=7.6 Hz, 2H), 6.93 (d, J=10 Hz, 4H). ¹³C NMR (400 MHz CDCl₃) δ 14.21, 19.08, 22.47, 25.6, 26.22, 27.46, 32.41, 40.12, 40.1, 43.88, 46.18, 127.44, 128.14, 136.25, 137.49, 141.58, 142.33.

5-[(4-N,N-(Dimethylamino)benzyl)-(4-{(2,4,6-trimethyl-benzenesulfonyl)-[3-(2,4,6-trimethyl-benzenesulfonylamino)propyl]amino}butyl)carbamoyl]pentanoic Acid Methyl Ester (46). Compound 46 was synthesized from 45 and 27a using the procedure described for the synthesis of 28d in 81.6% yield. ¹H NMR (400 MHz CDCl₃) δ 1.26 (m, 2H), 1.63 (m, 2H), 1.68 (m, 6H), 2.29 (m, 2H), 2.32 (m, 6H), 2.36 (m, 2H), 2.52 (m, 6H), 2.62 (m, 6H), 2.79 (m, 2H), 2.92 (s, 6H), 3.01-3.13 (m, 6H), 3.67 (s, 3H), 4.29 (s, 1H), 4.35 (s, 1H), 6.68 (d, J=8.4 Hz, 2H), 6.88-6.95 (m, 6H). IR (cm⁻¹) 3409.7, 2923.2, 1736.8, 1664.8, 1619.4, 1528.6.

5-[(4-N,N-(Dimethylamino)benzyl)-(4-{((2,4,6-trimethyl-benzenesulfonyl)-[3-(2,4,6-trimethyl-benzenesulfonylamino)propyl]amino}butyl)carbamoyl]pentanohydroxamic Acid (47). Compound 47 was synthesized in two steps from 46 using the procedures described for the syntheses of 29d and 30d in 66.2% overall yield. ¹H NMR (400 MHz D₂O) δ 1.40 (m, 4H), 1.50 (m, 2H) 1.63 (m, 4H) 1.80 (m, 1H), 1.98 (m, 1H), 2.07 (m, 1H), 2.31 (m, 1H), 2.71 (m, 1H), 2.90-2.93 (m, 8H), 3.14 (d, J=4.4 Hz, 6H), 3.38 (m, 2H), 7.31 (d, J=8.4 Hz, 2H), 7.44 (d, J=8.8 Hz, 1H), 7.49 (d, J=8.8 Hz, 1H). IR (cm⁻¹) 3398.3, 2941.8, 2683.2, 1613.0, 1522.2, 1470.3, 1184.9, 990.3.

5-[(4-N,N-(Dimethylamino)benzyl)-(4-{((2,4,6-trimethyl-benzenesulfonyl)-[3-(2,4,6-trimethyl-benzenesulfonylamino)propyl]amino}butyl)carbamoyl]pentanohydroxamic Acid (14). Compound 14 was synthesized in two steps from 47 using the procedure described for the synthesis of 11 in 82.2% yield. ¹H NMR (400 MHz D₂O) δ 1.14 (s, 9H), 1.43 (m, 4H), 1.59 (m, 4H), 1.72 (q, J=7.6 Hz, 2H), 2.11 (t, J=6.4 Hz, 2m), 2.32 (t, J=7.2 Hz, 2H), 2.85-2.94 (m, 6H), 3.12 (t, J=6.4 Hz, 2H), 4.05 (s, 2H), 7.27 (d, J=8.4 Hz, 2H), 7.41 (d, J=1.6 Hz, 2H). ¹³C NMR (400 MHz D₂O) δ 22.83, 22.96, 23.80, 24.05, 24.84, 25.77, 30.53, 33.49, 34.26, 35.43, 36.02, 45.17, 46.15, 46.97, 50.17, 126.44, 127.81, 129.92, 153.57, 177.28, 178.68. IR (cm⁻¹) 3396.8, 2929.7, 1677.8, 1606.5, 1424.9.

B. Inhibition of HDAC In Vitro

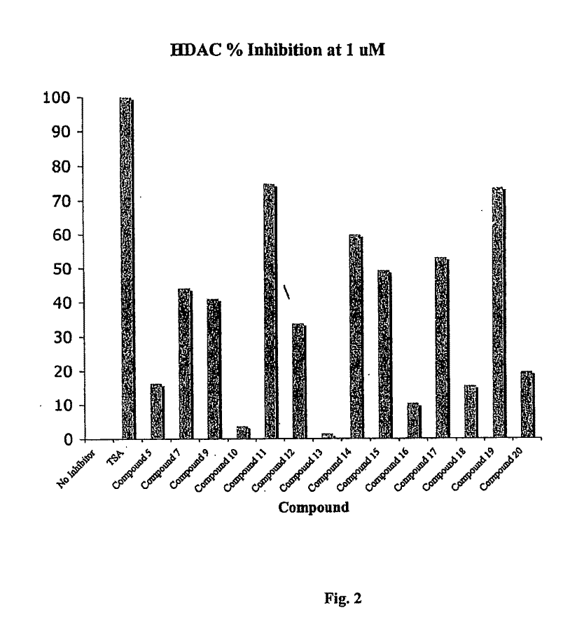

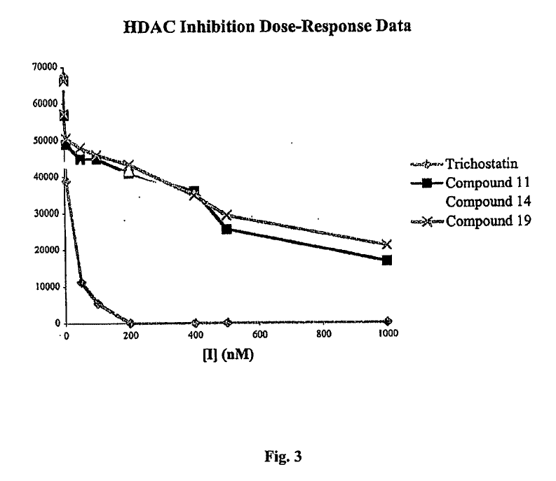

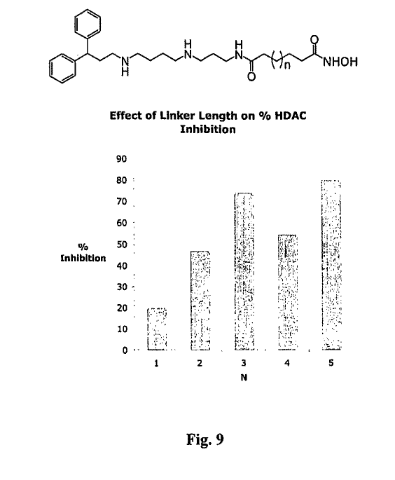

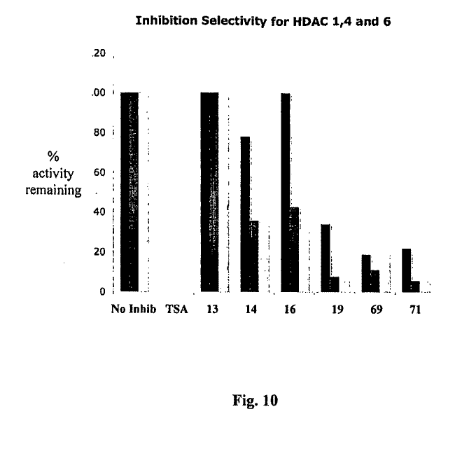

Compounds 5-20 were evaluated for their ability to inhibit isolated HDAC in a commercially available assay, employing TSA and MS-275 as positive controls. The results of these studies are summarized in Table 1, and in FIG. 2. Three of these analogues, compounds 11, 14 and 19, inhibited HDAC by 74.86, 59.99 and 73.85%, respectively. These three compounds were subjected to a dose response analysis using the same commercial assay kit, and varying the concentration of inhibitor between 0.5 and 1000 mM, with TSA as a positive control. As shown in FIG. 3, compounds 11, 14 and 19 were essentially equipotent over the concentration range tested. The level of HDAC inhibition with various homologues of compound 19 were measured as previously described (Pflum, et al., J. Biol. Chem. 2001, 276, 47733-47741). Compounds were evaluated for their ability to inhibit isolated HDAC at 1 μM in the Fluor de Lys™ assay system. Each data point is the average of three determinations that in each case differed by 3% or less. Results are in FIG. 9. Certain compounds are additionally test against specific HDAC homologs, with results shown in FIG. 10.

C. Cell Viability

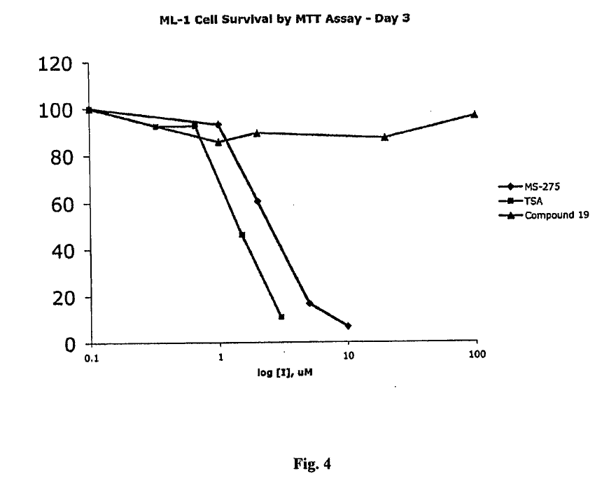

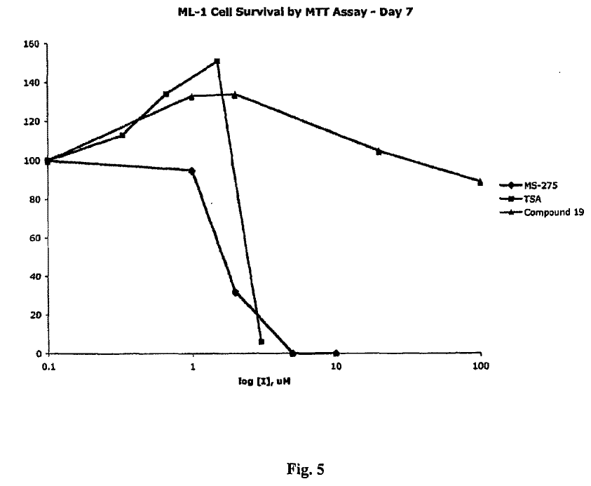

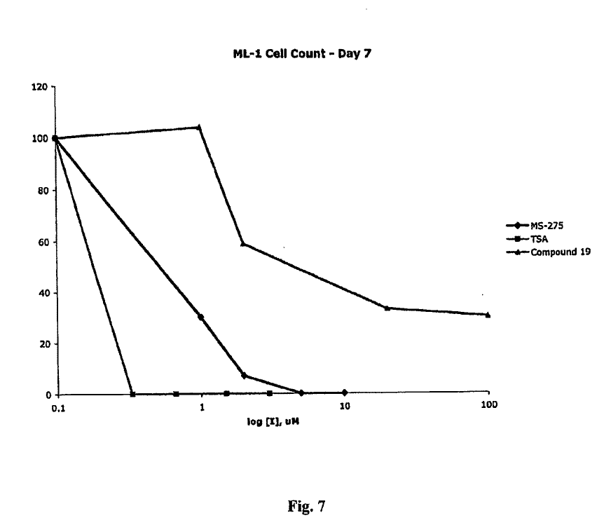

Compound 19 was evaluated in a series of cell viability studies in the ML-1 human myelocytic leukemia line. The results of these studies are outlined in FIGS. 4-7. Compound 19, as well as the positive controls TSA and MS-275, were compared at concentrations between 0.1 and 100 μM, and cell viability was determined in the ML-1 cultured cell preparation at 3 days (FIG. 4) and 7 days (FIG. 5) using a standard MTT assay. Significant toxicity was noted in the presence of TSA and MS-275, with IC₅₀ values less than 10 μM in all cases. By contrast, compound 19 did not produce significant toxicity at any of the concentrations tested at day 3 or 7. In a separate set experiments, cell viability was determined under the same conditions by direct cell count. As before, TSA and MS-275 produced significant toxicity at both 3 and 7 days, again with IC₅₀ values less than 10 μM. Compound 19 was again shown to be significantly less toxic, especially at concentrations greater than 10 μM, as shown in FIGS. 6 and 7.

D. Cellular Acetylation and Reexpression of p21

ML-1 cells after treatment were lysed in RIPA lysis buffer containing an EDTA-free protease inhibitor cocktail, at 4° C. for 30 min. Lysate was clarified by centrifugation at 14 000 rpm for 15 min. The resulting supernatant was used for analysis. The total protein content was determined by a bicinchoninic acid (BCA) assay kit (Pierce, Rockford, Ill.), and the absorbance of the solution was measured using a spectrophotometer at a wavelength of 570 nm. Absorbance was converted to protein content using an albumin standard curve.

The proteins (10 μg for histone or 30 μg for p₂₁^{waf1}) were separated by 15% SDS PAGE and visualized by Western blot analysis using the following antibodies against interesting proteins: antibodies for acetylhistone H3 (06-599) (diluted 1:1000), acetylhistone H4 (06-866) (diluted 1:500), and histone H2A (07-146) (diluted 1:1000) were from Upstate Biotechnologies, p₂₁^{Waf1} (556431) (diluted 1:500) from BD Pharmigen, and β-actin (ON365) (diluted 1:1000) from Oncogene Research Products. The immunoreactive proteins were detected using ECL western blotting analysis system (Amersham Biosciences, Piscataway, N.J.). Cell proliferation was quantified by the MTT assay according to the supplied protocol (Promega, Madison, Wis.).

Compound 19 was compared to MS-275 for the ability to promote hyperacetylation of histones H3 and H4 in the ML-1 cell line, as shown in FIG. 8. At 1 μM, compound 19 produced higher levels of acetylated H3 and H4 after 24 hours, as determined by Western blots derived from the immunoassay mixture described below. Histone H2a was also determined as an internal standard, and the levels of this protein did not change in the presence of 19 or MS-275. Reexpression of the tumor suppressor protein p21 was also determined in the presence of 19 and MS-275, and 19 was found to be effective at promoting the reexpression of this transcriptional product.

TABLE 1
Structure

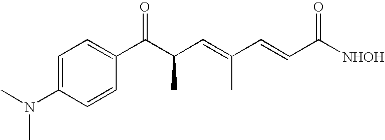

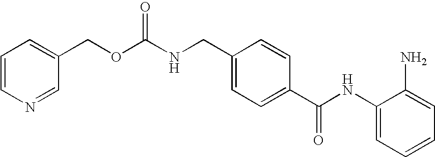

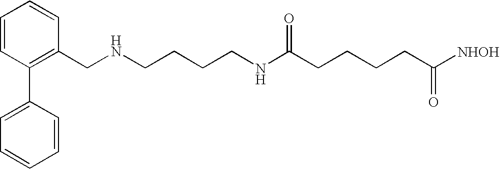

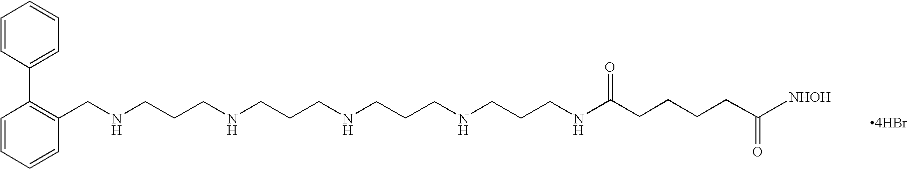

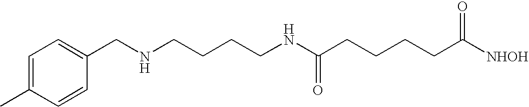

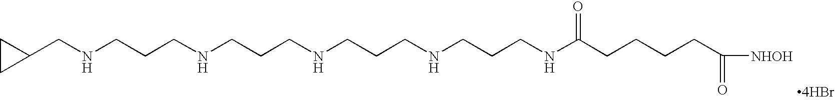

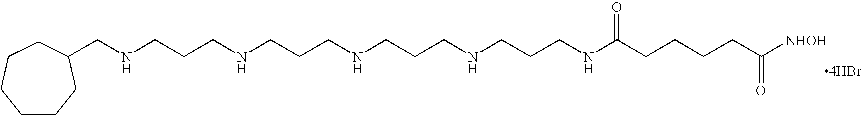

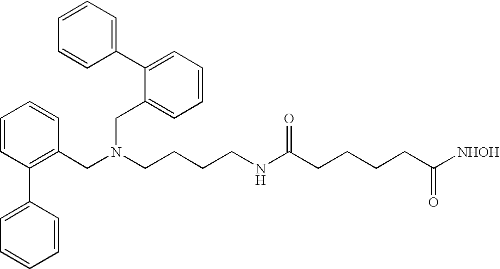

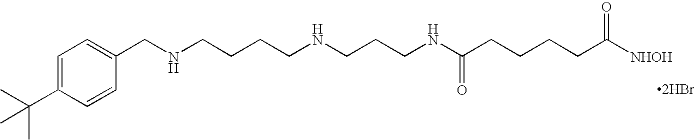

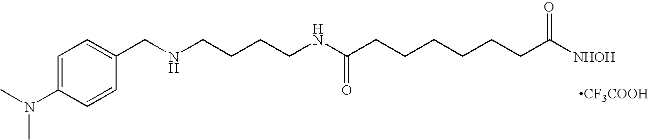

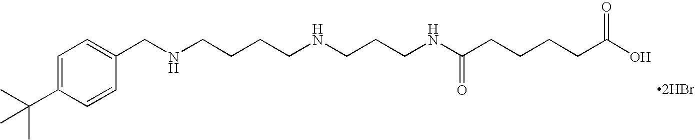

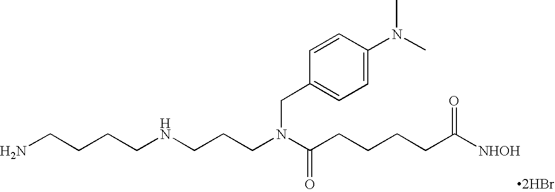

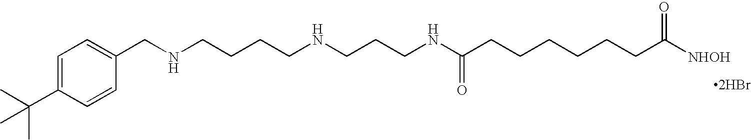

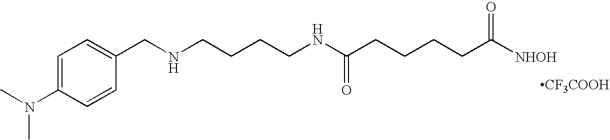

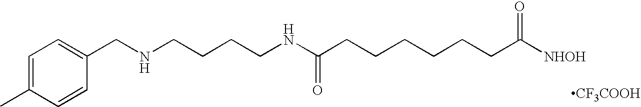

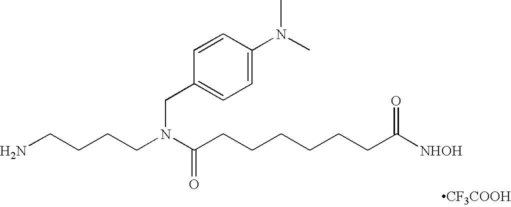

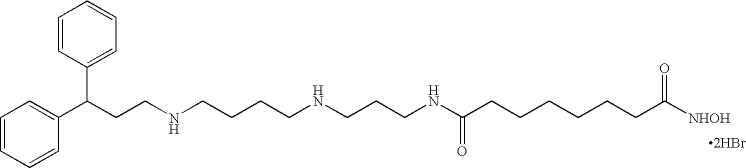

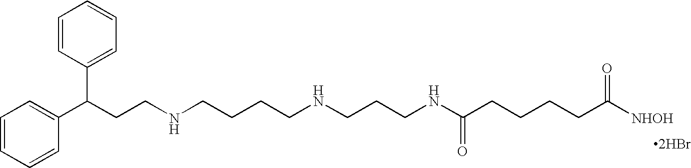

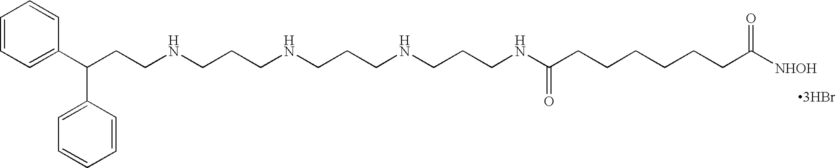

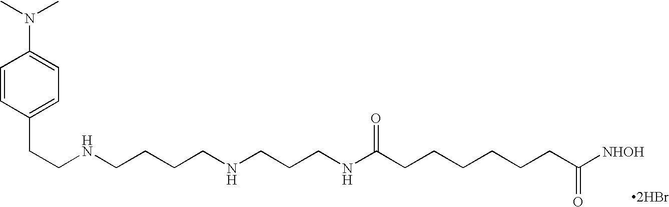

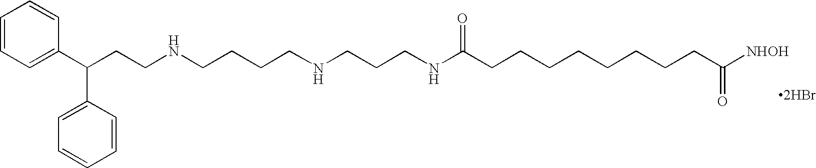

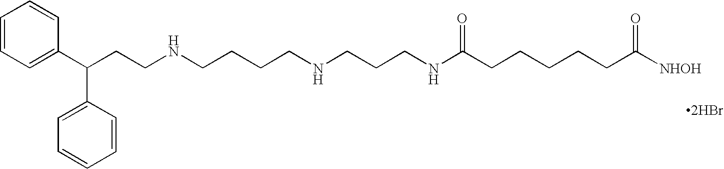

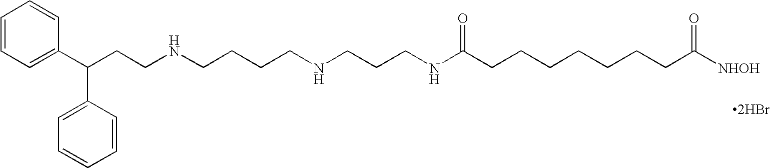

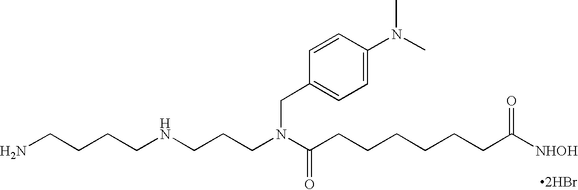

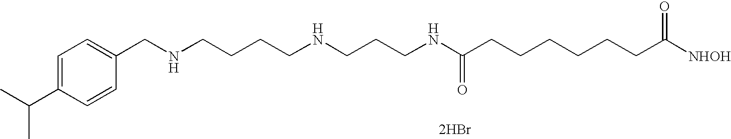

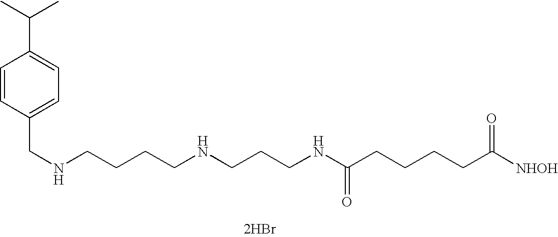

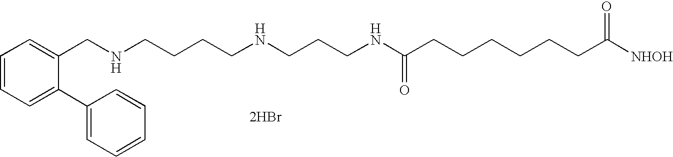

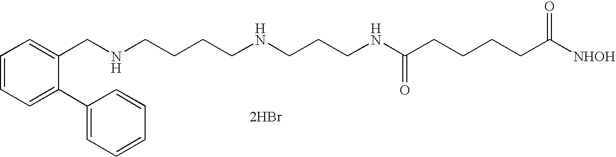

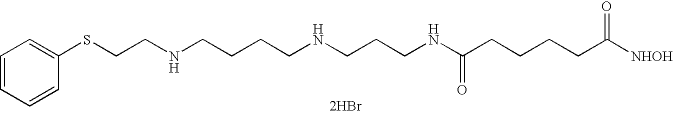

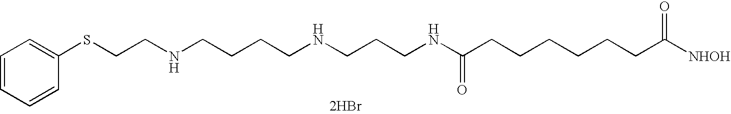

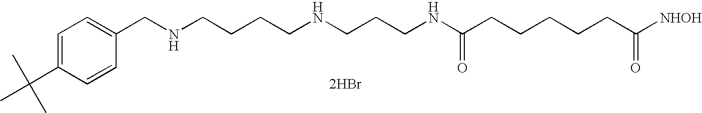

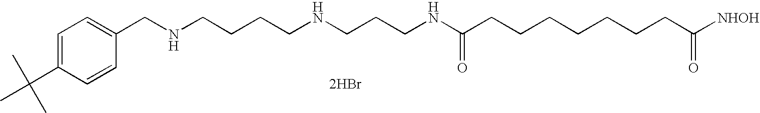

| | | | Inhibition |
|---|---|---|---|
| | | | of HDAC |
| | Empirical | | at 1 μM |
| No. | Formula | MW | (%) |
| 1 | C₁₇H₂₂NO₃ | 288.35 | 100% |
| | | | (control) |
| 2 | C₂₁H₂₀N₄O₃ | 376.41 | 100% |
| 5 | C₂₃H₃₁N₃O₃ | 397.51 | 16.34 |
| 6 | C₃₁H₅₄N₆O₃Br₄ | 1004.01 | |
| 7 | C₁₈H₂₉N₃O₃ | 335.44 | 44.17 |
| 8 | C₂₂H₅₀N₆O₃Br₄ | 766.29 | |
| 9 | C₂₆H₅₈N₆O₃Br₄ | 822.39 | 41.03 |
| 10 | C₂₆H₄₁N₃O₃ | 563.73 | 3.74 |
| 11 | C₂₂H₄₄N₄O₃Br₂ | 596.44 | 74.86 |
| 12 | C₂₃H₃₇N₄O₅F₃ | 508.56 | 33.89 |
| 13 | C₂₄H₄₃N₃O₃Br₂ | 581.42 | 1.48 |
| 14 | C₂₂H₄₁N₅O₃Br₂ | 583.40 | 59.99 |
| 15 | C₂₆H₄₇N₃O₃Br₂ | 609.48 | 49.36 |
| 16 | C₂₁H₃₃N₄O₅F₃ | 478.51 | 10.32 |
| 17 | C₂₂H₃₄N₃O₅F₃ | 477.52 | 53.2 |
| 18 | C₂₃H₃₇N₄O₅F₃ | 544.56 | 15.6 |
| 19 | C₃₀H₄₈N₄O₃Br₂ | 672.51 | 73.85 |
| 20 | C₂₈H₄₄N₄O₃Br₂ | 644.46 | 19.75 |
| 67 | C₃₂H₅₄Br₃N₅O₃ | 796.52 | 55 |
| 68 | C₂₅H₄₇Br₂N₅O₃ | 625.48 | 46 |
| 69 | C₃₂H₅₂Br₂N₄O₃ | 700.59 | 73 |
| 70 | C₂₉H₄₆Br₂N₄O₃ | 658.51 | 46 |
| 71 | C₃₁H₅₀Br₂N₄O₃ | 686.56 | 54 |
| 72 | C₂₄H₄₅Br₂N₅O₃ | 611.45 | 58 |
| 73 | C₂₅H₄₆Br₂N₄O₃ | 610.47 | |
| 74 | C₂₃H₄₂Br₂N₄O₃ | 582.41 | |
| 75 | C₂₈H₄₄Br₂N₄O₃ | 644.48 | |
| 76 | C₂₆H₄₀Br₂N₄O₃ | 616.43 | |
| 77 | C₂₁H₃₈Br₂N₄O₃S | 586.43 | |
| 78 | C₂₃H₄₂Br₂N₄O₃S | 614.48 | |
| 79 | C₂₅H₄₆Br₂N₄O₃ | 610.47 | |
| c | | | |
| 80 | C₂₇H₅₀Br₂N₄O₃ | 638.52 | |

What is claimed is:

1. A compound having the formula: 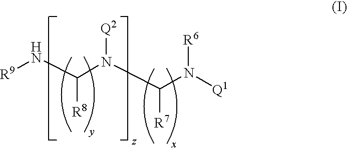
wherein
Q¹ and Q² are independently hydrogen or a moiety having the formula 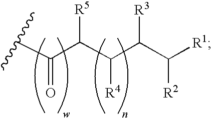
n is an integer from 1 to 20;
w is an integer from 0 to 1;
x and y are independently integers from 1 to 20;
z is an integer from 0 to 10;
R¹ is a Zn⁺²-binding moiety;
R², R³, R⁴, and R⁵ are independently hydrogen or unsubstituted C₁-C₆ alkyl;
R⁶ is hydrogen or -L¹-R¹⁰, wherein
L¹ is a bond, substituted or unsubstituted alkylene, or substituted or unsubstituted heteroalkylene, and
R¹⁰ is hydrogen, substituted or unsubstituted heterocycloalkyl, substituted or unsubstituted cycloalkyl, substituted or unsubstituted heteroaryl, or substituted or unsubstituted aryl;
R⁷ and R⁸ are independently hydrogen, —NH₂, or unsubstituted C₁-C₆ alkyl;
R⁹ is hydrogen or -L²-R¹¹, wherein
L² is a bond, substituted or unsubstituted alkylene, or substituted or unsubstituted heteroalkylene, and
R¹¹ is independently hydrogen, substituted or unsubstituted heterocycloalkyl, substituted or unsubstituted cycloalkyl, substituted or unsubstituted heteroaryl, or substituted or unsubstituted aryl;
wherein if z is 0 then Q¹ is not hydrogen, and if Q¹ is hydrogen then at least one Q² is not hydrogen and z is an integer from 1 to 10.

2. The compound of claim 1, wherein if R⁶ and R¹⁰ are hydrogen, then at least one of R⁹ or R¹¹ are not hydrogen, and if R⁹ and R¹¹ are hydrogen, then at least one of R⁶ or R¹⁰ are not hydrogen.

3. The compound of claim 1, wherein R¹ is —C(O)NHOH, —C(O)OH, —C(O)NH-(2-amino-phenyl), or substituted or unsubstituted tetrazolyl.

4. The compound of claim 1, wherein n is an integer from 1 to 5.

5. The compound of claim 1, wherein n is an integer from 2 to 5.

6. The compound of claim 1, wherein R², R³, R⁴, R⁵, R⁷ and R⁸ are hydrogen.

7. The compound of claim 1, wherein w is 1.

8. The compound of claim 1, wherein z is an integer from 1 to 2.

9. The compound of claim 1, wherein x and y are independently integers from is 2 to 5.

10. The compound of claim 1, wherein x and y are independently integers from is 3 to 4.

11. The compound of claim 1, wherein x is 3 and y is independently 4.

12. The compound of claim 1, wherein R⁶ is hydrogen and R⁹ is -L²-R¹¹, wherein
L² is substituted or unsubstituted alkylene, and
R¹¹ is substituted or unsubstituted aryl.

13. The compound of claim 12, wherein L² is substituted or unsubstituted C₁-C₅ alkylene.

14. The compound of claim 12, wherein L² is unsubstituted C₁-C₈ alkylene or C₁-C₅ alkylene substituted with substituted or unsubstituted aryl.

15. The compound of claim 12, wherein L² is unsubstituted C₁-C₅ alkylene or C₁-C₅ alkylene substituted with unsubstituted aryl.

16. The compound of claim 12, wherein L² is —CH₂—CH₂—CH(phenyl)-.

17. The compound of claim 12, wherein R¹¹ is unsubstituted aryl or aryl substituted with unsubstituted C₁-C₁₀ alkyl, unsubstituted aryl, or —NR²R³, wherein R¹² and R¹³ are independently hydrogen or unsubstituted C₁-C₁₀ alkyl.

18. The compound of claim 1, wherein only one of Q¹ or Q² is not hydrogen.

19. A method of treating cancer in a subject in need thereof comprising administering to the subject a therapeutically effective amount of the compound of claim 1.

20. A pharmaceutical formulation comprising the compound of claim 1 and a pharmaceutically acceptable excipient.

21. A method of decreasing the catalytic activity of a histone deacetylase comprising contacting said histone deacetylase with the compound of claim 1.
